(19) United States
(12) Patent Application Publication
(10) Pub. No.: US 2024/0024507 A1
(43) Pub. Date: Jan. 25, 2024

(54) NOVEL COMPOSITIONS WITH TISSUE-SPECIFIC TARGETING MOTIFS AND COMPOSITIONS CONTAINING SAME

(71) Applicant: The Trustees of the University of Pennsylvania, Philadelphia, PA (US)

(72) Inventors: James M. Wilson, Philadelphia, PA (US); Joshua Joyner Sims, Philadelphia, PA (US); Yuan Yuan, Boston, MA (US)

(73) Assignee: The Trustees of the University of Pennsylvania, Philadelphia, PA (US)

(21) Appl. No.: 18/255,154

(22) PCT Filed: Dec. 1, 2021
(86) PCT No.: PCT/US2021/061312

§ 371 Date: May 31, 2023

Related U.S. Application Data
(60) Provisional application No. 63/119,863, filed on Dec. 1, 2020.

(51) Int. Cl. A61K 48/00; C12N 15/86; A61K 39/42; A61K 39/215; A61K 38/17; A61P 31/14

(52) U.S. Cl. CPC A61K 48/0041; C12N 15/86; A61K 39/42; A61K 39/215; A61K 38/1709; A61K 48/0066; A61P 31/14; C12N 2750/14122; C12N 2750/14143; C12N 2750/14145; C12N 2750/14171; A61K 2039/5256

(57) ABSTRACT

Provided herein are compositions including targeting peptides linked thereto or inserted in a targeting protein of a recombinant vector having at least one exogenous peptide comprising an amino acid sequence of N-x-(T/I/V/A)-(K/R) (SEQ ID NO: 47). Compositions providing such conjugates, targeting peptides, or recombinant vectors having a mutant capsid or envelope protein are provided as are uses thereof.

### BACKGROUND OF THE INVENTION

The Adeno-Associated Virus (AAV) is currently the gene therapy vector of choice. This is because AAVs can deliver a transgene that is stably expressed for decades from a non-integrating genome, and because the AAV is remarkably safe and non-immunogenic. However, AAV gene therapy is currently limited to a small number of diseases due to challenges in delivery and tropism. This is particularly true for disorders of the central nervous system (CNS). Direct delivery of AAV gene therapy vectors is possible, by injecting the vector directly into the cerebrospinal Fluid (CSF), but this method typically transduces 1% or less of brain cells. Furthermore, most of that transduction is concentrated on the cells that are in direct contact with the CSF. Cells in the “deep brain” are rarely transduced. This has limited the number of CNS disorders treatable by gene therapy.

In contrast to the CSF network, the vascular system of the brain reaches nearly every cell in the CNS. This is because of a high demand these tissues have for glucose, oxygen, and other nutrients. However, cells in the brain and spinal cord are protected from the circulatory system by a specialized vascular unit, the Blood Brain Barrier (BBB). The BBB limits the diffusion of large molecules like viral vectors and proteins, and even many small molecule drugs through a complex network of tightly-linked cells that surround the blood vessels of the brain and spinal cord. Thus, a grand challenge in gene therapy delivery to the CNS has been the engineering of an AAV variant capable of crossing the BBB at high efficiency and transducing cells in the deep brain.

One AAV capsid developed at California Institute of Technology (CalTech) has a seven amino acid peptide inserted into hypervariable loop 8 (HVR8) on the AAV9 capsid to generate a rAAV called AAV9-PHP.B which is reported to mediates interaction with Ly6a, a GPI-anchored receptor on the brain vasculature of some mouse strains. US patenttent Published Application No. 2017/0166926A1. This interaction drives transport of AAV9-PHP.B across the BBB, resulting in ˜50-fold higher transduction of brain cells than AAV9. However, this finding has not translated to larger animals or humans.

There remains a need for vectors which can specifically target selected tissue and cell types.

### SUMMARY OF THE INVENTION

In certain embodiments, a recombinant adeno-associated virus particle (rAAV) having a capsid comprising an amino acid sequence that comprises the motif N-x-(T/I/V/A)-(K/R) (SEQ ID NO: 47) is provided. Suitably, the amino acid sequence is part of at least the AAV vp3 protein in the capsid and a vector genome packaged in the capsid which comprises a nucleic acid sequence encoding a gene product under control of sequences which direct expression thereof, provided that the capsid is not a mutant AAV2 capsid comprising an NDVRAVS (SEQ ID NO: 48) sequence. In certain embodiments, the amino acid sequence comprises comprising the N-x-(T/I/V/A)-(K/R) motif optionally flanked at the amino terminus and/or the carboxy terminus of the motif by two amino acids to seven amino acids is inserted into the AAV capsid vp3 region. In certain embodiments, the sequence inserted into the capsid comprises: (a) SSNTVKLTSGH (SEQ ID NO: 40); (b) EFSSNTVKLTS (SEQ ID NO: 38); (c) GGVLTNIARGEYMRGG (SEQ ID NO: 46); (d) GGIEINATRAGTNLGG (SEQ ID NO: 43); (e) GGSSNTVKLTSGHGG (SEQ ID NO: 39); (f) IEINATRAGTNL (SEQ ID NO: 42); or (g) SANFIKPTSY (SEQ ID NO: 41) In certain embodiments, the amino acid sequence of the motif is NTVK, which is optionally flanked by two to seven amino acids at its carboxy—and/or amino terminus and inserted between amino acids 588 and 589 of an AAV9 capsid protein, based on the numbering of amino acid sequence: SEQ ID NO: 44.

In certain embodiments, a rAAV having an inserted sequence of NTVK in its capsid, which sequence is optionally flanked by two to seven amino acids at its carboxy- and/or amino terminus and inserted between amino acids 588 and 589 of an AAV9 capsid protein, based on the numbering of amino acid sequence: SEQ ID NO: 44.

In certain embodiments, a composition comprises the rAAV having the inserted motif and optional flanking sequences and one or more of a physiologically compatible carrier, excipient, and/or aqueous suspension base.

In certain embodiments, an endothelial cell targeting peptide is provided, the peptide comprising an amino acid sequence of N-x-(T/I/V/A)-(K/R) (SEQ ID NO: 47) optionally flanked at the amino terminus and/or the carboxy terminus of the motif by two amino acids to seven amino acids, and optionally further conjugated to a nanoparticle, a second molecule, or a viral capsid protein. In certain embodiments, the endothelial cell targeting peptide comprises: (a) SSNTVKLTSGH (SEQ ID NO: 40); (b) EFSSNTVKLTS (SEQ ID NO: 38); (c) GGVLTNIARGEYMRGG (SEQ ID NO: 46); (d) GGIEINATRAGTNLGG (SEQ ID NO: 43); (e) GGSSNTVKLTSGHGG (SEQ ID NO: 39); (f) IEINATRAGTNL (SEQ ID NO: 42); or (g) SANFIKPTSY(SEQ ID NO: 41). In certain embodiments, the amino acid sequence of the motif is NTVK. In certain embodiments, a composition is provided which comprises the endothelial cell targeting peptide and one or more of a physiologically compatible carrier, excipient, and/or aqueous suspension base.

In certain embodiments, a fusion polypeptide or protein comprising a brain endothelial cell targeting peptide and a fusion partner which comprises at least one polypeptide or protein is provided herein. In certain embodiments, a composition comprising a fusion polypeptide or protein according to claim 11 and one or more of a physiologically compatible carrier, excipient, and/or aqueous suspension base.

Provided herein are compositions and methods for using an rAAV, an endothelial cell targeting peptide, a fusion polypeptide or protein, and/or a composition as described herein of for delivering a therapeutic to a patient in need thereof. In certain embodiments, the therapeutic is targeted to the brain endothelial cells.

In certain embodiments, a composition and method is provided for treating Allan-Herndon-Dudley disease by delivering to a subject in need thereof an rAAV as described herein wherein the encoded gene product is an MCT8 protein.

In certain embodiments, a method is provided for targeting therapy to the lung comprising administering to a patient in need thereof an rAAV as described herein.

In certain embodiments, a method is provided for treating a disease of the lung by delivering to a subject in need thereof an rAAV having a capsid with the inserted targeting peptide and encoding a therapeutic gene product, wherein the encoded gene product is a soluble Ace2 protein, an anti-SARS antibody, an anti-SARS-CoV2 antibody, an anti-influenza antibody, or a cystic fibrosis transmembrane protein.

In certain embodiments, a method is provided for increasing transduction of AAV production cells in vitro comprising inserting an N-x-(T/I/V/A)-(K/R) motif into an AAV capsid. In certain embodiments, the production cells are 293 cells.

These and other embodiments and advantages of the invention will be apparent from the specification, including, without limitation, the detailed description of the invention.

### BRIEF DESCRIPTION OF THE FIGURES

FIGS. 1A to 1B shows the enrichment scores for the top performing peptide hits in mouse brain in the screen, with reference peptides. FIG. 1A shows enrichment scores for C57BL/6J mice. FIG. 1B shows enrichment scores for Balb/c mice.

FIGS. 2A and 2B show the enrichment scores for the top performing hits in NHP tissue in the screen. FIG. 2A shows enrichment scores for NHP brain. FIG. 2B shows enrichment scores for NHP spinal cord tissue.

FIGS. 3A to 3D show a secondary validation of the transduction levels of top performing peptide hits in AAV capsid comprising GFP reporter transgene. The results are plotted relative to AAV9 transduction. FIG. 3A shows secondary validation screen of selected peptide targeting of brain tissue in Balb/c mice. FIG. 3B shows secondary validation screen of selected peptide targeting of brain tissue in C57BL/6 mice. FIG. 3C shows secondary validation screen of selected peptide targeting of liver tissue in Balb/c mice. FIG. 3D shows secondary validation screen of selected peptide targeting of liver tissue in C57BL/6 mice.

FIG. 4 shows region of the alignment of the amino acid sequences of the various AAV capsid proteins of AA9, AAV8, AAV7, AAV6, AAV5, AAV4, AAV3B. AAV2, and AAV1, which is focused on the region HVRVIII in which the targeting peptide may be inserted (based on structure analysis).

FIG. 5 shows that “NxTK” motif is the critical motif for brain biodistribution in the SAN insert, and shows average impact of substitution (fold-change from original sequence).

FIG. 6 show that “NxTK” motif controls plasmid-to-AAV conversion in the SAN peptide insert, and shows average impact of substitution (fold-change from original sequence).

FIGS. 7A to 7D show that “NxTK” motif confers broad transduction advantage across cell lines. FIG. 7A shows relative transduction levels when compared to AAV9 capsid in 293 cells. FIG. 7B shows relative transduction levels when compared to AAV9 capsid in NIH3T3 cells. FIG. 7C shows relative transduction levels when compared to AAV9 capsid in HUH7 cells. FIG. 7D shows transduction levels at day 3 post-transduction (3DPT) and day 7 post transduction (7DPT) in macaque primary airway cells. FIG. 7E shows microscopic analysis of the macaque primary airway epithelial cells in a control sample treated with carrier (i.e., no vector). FIG. 7F shows microscopic analysis of the macaque primary airway epithelial cells post-transduction with AAV9-GFP vector. FIG. 7G shows microscopic analysis of the macaque primary airway epithelial cells post-transduction with AAV9-GFP vector comprising EFS peptide insert. FIG. 7H shows microscopic analysis of the macaque primary airway epithelial cells post-transduction with AAV9-GFP comprising SAN peptide inserts.

FIG. 8 shows a preliminary transduction test with GFP vectors in cultured human cells (nasal, bronchial and tracheal) plotted as a ratio of mRNA copy number over micro-gram total mRNA.

### DETAILED DESCRIPTION OF THE INVENTION

A targeting peptide sequence is provided herein. Also provided herein are fusion proteins, modified proteins, mutant viral capsids and other moieties operably linked to an exogenous targeting peptide motif of N-x-(T/I/V/A)-(K/R) (SEQ ID NO: 47). In certain embodiments, this exogenous motif confers on these compositions a modifies the native tissue specificity of the source (parental) protein, viral vector, or other moiety. In certain embodiments, targeting peptides in this motif provides enhanced or altered endothelial cell targeting. In certain embodiments, targeting peptides in this motif provide enhanced or altered lung, bronchial, tracheal and/or nasoepithelial targeting. In certain embodiments, viral vectors having modified capsids with this motif exhibit increased transduction of AAV production cells in vitro.

The targeting peptide may be linked to a recombinant protein (e.g., for enzyme replacement therapy) or polypeptide (e.g., an immunoglobulin) to target to the desired tissue (e.g., CNS or lung) to form a fusion protein or a conjugate. Additionally, the targeting peptide may be linked to a liposome and/or a nanoparticle (a lipid nanoparticle, LNP) forming a peptide-coated liposome and/or LNP to target the desired tissue. Sequences encoding at least one copy of a targeting peptide and optional linking sequences may be fused in frame with the coding sequence for the recombinant protein and co-expressed with the protein or polypeptide to provide fusion proteins or conjugates. Alternatively, other synthetic methods may be used to form a conjugate with a protein, polypeptide or another moiety (e.g., DNA, RNA, or a small molecule). In certain embodiments, multiple copies of a targeting peptide are in the fusion protein/conjugate. Suitable methods for conjugating a targeting peptide to a recombinant protein include modifying the amino (N)-terminus and one or more residues on a recombinant human protein (e.g., an enzyme) using a first crosslinking agent to give rise to a first crosslinking agent modified recombinant human protein, modifying the amino (N)-terminus of a short extension linker region preceding a targeting peptide using a second crosslinking agent to give rise to a second crosslinking agent modified variant target peptide, and then conjugating the first crosslinking agent modified recombinant human protein to the second crosslinking agent modified variant targeting peptide containing a short extension linker. Other suitable methods of conjugating a targeting peptide to a recombinant protein include conjugating a first crosslinking agent modified recombinant human protein to one or more second crosslinking agent modified variant targeting peptides, wherein the first crosslinking agent modified recombinant protein comprises a recombinant protein characterized as having a chemically modified N-terminus and one or more modified lysine residues and the one or more second crosslinking agent modified variant targeting peptides comprise one or more variant targeting peptides comprising a modified N-terminal amino acid of a short extension linker preceding the targeting peptide. Still other suitable methods for conjugating a targeting peptide to a protein, polypeptide, nanoparticle, or another biologically useful chemical moiety may be selected. See, e.g., U.S. Pat. No. 9,545,450 B2 (NHS-phosphine cross-linking agents; NHS-Azide cross-linking agents); US Published patent application No. US 2018/0185503 A1 (aldehyde-hydrazide crosslinking).

In certain embodiments, the targeting peptide may be inserted into a suitable site within a protein or polypeptide (e.g., a viral capsid protein). In certain these embodiments and in certain other embodiments, a targeting peptide may be flanked at its carboxy (COO—) and/or amino (N) terminus by a short extension linker. Such a linker may be 1 to 20 amino acid residues in length, or about 2 to 20 amino acids residues, or about 1 to 15 amino acid residues, or about 2 to 12 amino acid residues, or 2 to 7 amino acid residues in length. The short extension linker can also be about 10 amino acids in length. The presence and length of a linker at the N-terminus is independently selected from a linker at the carboxy-terminus, and the presence and length of a linker at the carboxy terminus is independently selected from a linker at the N-terminus. Suitable short extension linkers can be provided using a 5-amino acid flexible GS extension linker (glycine-glycine-glycine-glycine-serine), a 10-amino acid extension linker comprising 2 flexible GS linkers, a 15-amino acid extension linker comprising 3 flexible GS linkers, a 20-amino acid extension linker comprising 4 flexible GS linkers, or any combination thereof.

In certain embodiment, a composition is provided which is useful for targeting an endothelial cell. The composition is a mutant capsid, fusion protein or another conjugate comprising at least one exogenous targeting peptide comprising: an amino acid sequence of N-x-(T/I/V/A)-(K/R) (SEQ ID NO: 47) optionally flanked at the amino terminus and/or the carboxy terminus of the motif by two amino acids to seven amino acids, and optionally further conjugated to a nanoparticle, a second molecule, or a viral capsid protein. The targeting peptide comprising the following sequence with optional linking sequences:

(a)
(SEQ ID NO: 40)
SSNTVKLTSGH;
(b)
(SEQ ID NO: 38)
EFSSNTVKLTS;
(c)
(SEQ ID NO: 46)
GGVLTNIARGEYMRGG;
(d)
(SEQ ID NO: 43)
GGIEINATRAGTNLGG;
(e)
(SEQ ID NO: 39)
GGSSNTVKLTSGHGG;
(f)
(SEQ ID NO: 42)
IEINATRAGTNL;
or
(g)
(SEQ ID NO: 41)
SANFIKPTSY.

In certain embodiments, the targeting peptide motif is encoded by a nucleic acid sequence selected from:

(a)
(SEQ ID NO: 54)
agcagcaacaccgtgaagctgaccagcggacac;
(b)
(SEQ ID NO: 50)
gagttcagcagcaacaccgtgaagctgaccagc;
(c)
(SEQ ID NO: 56)
ggaggagtgctgaccaacatcgctagaggagagtacatgagaggagga;
(d)
(SEQ ID NO: 52)
ggaggaatcgagatcaacgctaccagagctggaaccaacctgggagga;
(e)
(SEQ ID NO: 55)
ggaggaagcagcaacaccgtgaagctgaccagcggacacggagga;
(f)
(SEQ ID NO: 51)
atcgagatcaacgctaccagagctggaaccaacctg;
or
(g)
(SEQ ID NO: 53)
agcgctaacttcatcaagcctaccagctac.

In certain embodiments, the targeting peptide is encoded by a nucleic acid sequence of SEQ ID NO: 50, or a sequence at least about 70% identical thereto. In certain embodiments, the targeting peptide is encoded by a nucleic acid sequence of SEQ ID NO: 51, or a sequence at least about 70% identical thereto. In certain embodiments, the targeting peptide is encoded by a nucleic acid sequence of SEQ ID NO: 52, or a sequence at least about 70% identical thereto. In certain embodiments, the targeting peptide is encoded by a nucleic acid sequence of SEQ ID NO: 53, or a sequence at least about 70% identical thereto. In certain embodiments, the targeting peptide is encoded by a nucleic acid sequence of SEQ ID NO: 54, or a sequence at least about 70% identical thereto. In certain embodiments, the targeting peptide is encoded by a nucleic acid sequence of SEQ ID NO: 55, or a sequence at least about 70% identical thereto. In certain embodiments, the targeting peptide is encoded by a nucleic acid sequence of SEQ ID NO: 56, or a sequence at least about 70% identical thereto. In some embodiments, the nucleic acid sequence encoding for the targeting peptide motif is optionally flanked at the 5′ and/or 3′ ends of the nucleic acid sequence of the motif by six to twenty one nucleotides of an extension linker.

In certain embodiments, the targeting peptide NTVK. In certain embodiments, the targeting peptide NTVR. In certain embodiment, more than one copy of a targeting peptide within this motif is provided in a conjugate or modified protein (e.g., a parvovirus capsid). In certain embodiments, two or more different targeting peptides are present

In certain embodiment, a composition is provided which is useful for targeting a nasoepithelial and/or lung epithelial cell. The composition is a mutant capsid, fusion protein or another conjugate comprising at least one exogenous targeting peptide comprising: an amino acid sequence of N-x-(T/I/V/A)-(K/R) (SEQ ID NO: 47) optionally flanked at the amino terminus and/or the carboxy terminus of the motif by two amino acids to seven amino acids, and optionally further conjugated to a nanoparticle, a second molecule, or a viral capsid protein. The targeting peptide comprises: (a) SSNTVKLTSGH (SEQ ID NO: 40); (b) EFSSNTVKLTS (SEQ ID NO: 38); (c) GGVLTNIARGEYMRGG (SEQ ID NO: 46); (d) GGIEINATRAGTNLGG (SEQ ID NO: 43); (e) GGSSNTVKLTSGHGG (SEQ ID NO: 39); (f) IEINATRAGTNL (SEQ ID NO: 42); or (g) SANFIKPTSY (SEQ ID NO: 41).

In certain embodiments, the targeting peptide NTVK. In certain embodiments, the targeting peptide NTVR, optionally flanked by spacer amino acids as described herein. In certain embodiment, more than one copy of a targeting peptide within this motif is provided in a conjugate or modified protein (e.g., a parvovirus capsid). In certain embodiments, two or more different targeting peptides are present.

Examples of suitable proteins, including enzymes, immunoglobulins, therapeutic proteins, immunogenic polypeptides, nanoparticles, DNA, RNA, and other moieties (e.g., small molecules, etc.) for targeting are described in more detail below. These and other biologic and chemical moieties are suitable for use with the targeting peptide(s) provided herein.

In certain embodiments, a composition is a nucleic acid sequence molecule, wherein the nucleic acid sequence is a DNA molecule or RNA molecule, e.g., naked DNA, naked plasmid DNA, messenger RNA (mRNA), containing the targeting peptide sequence motif linked to the nucleic acid molecule. In some embodiments, the nucleic acid molecule is further coupled with various compositions and nano particles, including, e.g., micelles, liposomes, cationic lipid—nucleic acid compositions, poly-glycan compositions and other polymers, lipid and/or cholesterol-based—nucleic acid conjugates, and other constructs such as are described herein. See, e.g., WO2014/089486, US 2018/0353616A1, US2013/0037977A1, WO2015/074085A1, U.S. Pat. No. 9,670,152B2, and U.S. Pat. No. 8,853,377B2, X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference. In certain embodiments, the targeting peptide motif is chemically linked to a nanoparticle surface, wherein the nanoparticle encapsulates a nucleic acid molecule. In some embodiments the nanoparticle comprising the targeting peptide linked to the surface is designed for targeted tissue-specific delivery. In some embodiments two or more different targeting peptides are linked to the surface of the nanoparticle. Suitable chemical linking or cross-linking include those known to one skilled in the art.

### Capsids

In certain embodiments, a recombinant parvovirus is provided which has a modified parvovirus capsid having at least exogenous peptide from the N-x-(T/I/V/A)-(K/R) targeting motif. Such a recombinant parvovirus may be a hybrid bocavirus/AAV or a recombinant AAV vector. In other embodiments, other viral vectors may be generated having one or more exogenous targeting peptides from the N-x-(T/I/V/A)-(K/R) motif (which may be same or different, or combinations thereof) in an exposed capsid protein to modulate and/or alter the targeting specificity of the viral vector as compared to the parental vector.

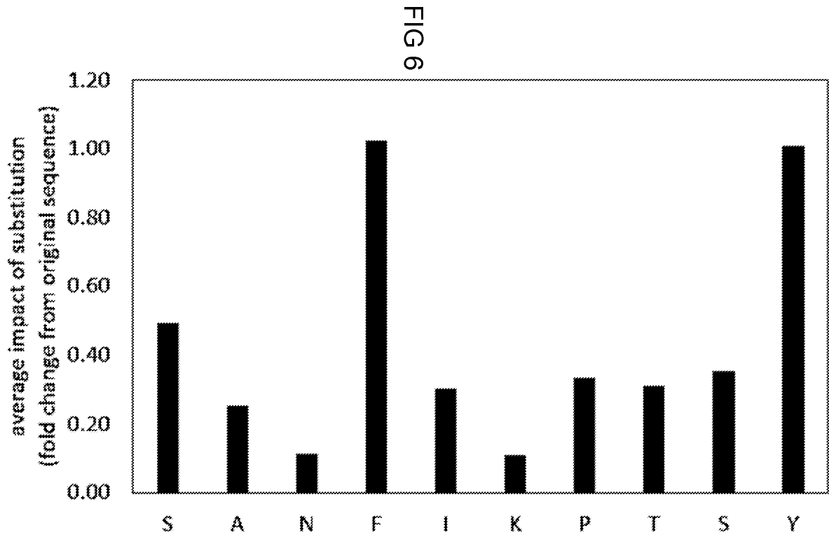

The targeting peptide may be inserted into a hypervariable loop (HVR) VIII at any suitable location. For example, based on the numbering of the AAV9 capsid, the peptide is inserted with linkers of various lengths between amino acids 588 and 589 (Q-A) of the AAV9 capsid protein, based on the numbering of the AAV9 VP1 amino acid sequence: SEQ ID NO: 44. See, also, WO 2019/168961, published Sep. 6, 2019, including Table G providing the deamidation pattern for AAV9 and WO 2020/160582, filed Sep. 7, 2018. The amino acid residue locations are identical in AAVhu68 (SEQ ID NO: 45). However, another site may be selected within HVRVIII. Alternatively, another exposed loop HVR (e.g., HVRIV) may be selected for the insertion. Comparable HVR regions may be selected in other capsids. In certain embodiments, the location for the HVRVIII and HVRIV is determined using an algorithm and/or alignment technique as described in U.S. Pat. No. 9,737,618 B2 (column 15, lines 3-23), and U.S. Pat. No. 10,308,958 B2 (column 15, line 46—column 16, line 6), which are incorporated herein by reference in its entirety. In certain embodiments, AAV1 capsid protein is selected as a parental capsid, wherein the targeting peptide with linkers of various lengths is inserted in a suitable location of the HVRVIII region of amino acid 582 to 585, or HVRIV region of amino acid 456 to 459 based on vp1 numbering (Gurda, B L., et al., Capsid Antibodies to Different Adeno-Associated Virus Serotypes Bind Common Regions, 2012, Journal of Virology, Jun. 12, 2013, 87(16): 9111-91114). In certain embodiments, AAV8 is selected as parental capsid, wherein the targeting peptide with linkers of various length is inserted in a suitable location of HVRVIII region of amino acid 586 to 591 (e.g., 590-591 (N-T)), or HVRIV region of amino acid 456 to 460, based on VP1 numbering (Gurda, B L., et al., Mapping a Neutralizing epitope onto the Capsid of Adeno-Associated Virus Serotype 8, 2012, Journal of Virology, May 16, 2012, 86(15):7739-7751). In certain embodiments, the AAV7 is selected as parental capsid, wherein the targeting peptide with linkers of various length is inserted in a suitable location of amino acid 589 to 590 (N-T). In certain embodiments, the AAV6 is selected as parental capsid, wherein the targeting peptide with linkers of various length is inserted in a suitable location of amino acid 588 to 589 (S-T). In certain embodiments, the AAV5 is selected as parental capsid, wherein the targeting peptide with linkers of various length is inserted in a suitable location of amino acid 577 to 578 (T-T). In certain embodiments, the AAV4 is selected as parental capsid, wherein the targeting peptide with linkers of various length is inserted in a suitable location of amino acid 586 to 587 (S—N). In certain embodiments, the AAV3B is selected as parental capsid, wherein the targeting peptide with linkers of various length is inserted in a suitable location of amino acid 588 to 589 (N-T). In certain embodiments, the AAV2 is selected as parental capsid, wherein the targeting peptide with linkers of various length is inserted in a suitable location of amino acid 587 to 588 (N—R). In certain embodiments, the AAV1 is selected as parental capsid, wherein the targeting peptide with linkers of various length is inserted in a suitable location of amino acid 589 to 589 (S-T). See also, FIG. 4.

In certain embodiments, the parental capsid modified to contain the N-x-(T/I/V/A)-(K/R) motif, with optional flanking sequences, is selected from parvoviruses which natively target the CNS (e.g., Clade F AAV (e.g., AAVhu68 or AAV9), Clade E (e.g., AAV8), or certain Clade A AAV (e.g., AAV1, AAVrh91)) capsids, or non-parvovirus capsids (e.g., herpes simplex virus, etc.) in order enhance expression and/or otherwise modulate the type of CNS targeted cells. In other embodiments, the capsid is selected from parvoviruses which do not natively target the CNS (e.g., Clade F AAV, e.g., AAVhu68 or AAV9, or certain Clade A AAV, e.g., AAV1, AAVrh91) capsids, or non-parvovirus capsids (e.g., herpes simplex virus (HSV), etc.). See, e.g., WO 2020/223231, published Nov. 5, 2020 (rh91, including table with deamidation pattern), U.S. Provisional patent application No. 63/065,616, filed Aug. 14, 2020 and U.S. Provisional patent application No. 63/109,734, filed Nov. 4, 2020. In certain embodiments, the capsid is selected form AAV Clade F AAVhu95 and AAVhu96 capsids. See, e.g., U.S. Provisional Application No. 63/251,599, filed Oct. 2, 2201.

In certain embodiments, the parental capsid modified to contain the N-x-(T/I/V/A)-(K/R) motif is selected from viruses (e.g., AAV) which natively target nasal epithelial cells, nasopharynx cells, and/or lung cells in order to enhance targeting as compared to the parental AAV (e.g., Clade A AAV, e.g., AAV1, AAVrh32.33, AAV6.2, AAV6, AAVrh91), or AAV5, or certain Clade F AAV, e.g., AAVhu68 or AAV9, capsids, or non-parvovirus capsids (e.g., adenoviruses, HSV, RSV, etc.). See, e.g., WO 2020/223231, published Nov. 5, 2020 (rh91, including table with deamidation pattern), U.S. Provisional patent application No. 63/065,616, filed Aug. 14, 2020 and U.S. Provisional patent application No. 63/109,734, filed Nov. 4, 2020.

In certain embodiments, the AAV capsid is not a mutant AAV2 capsid comprising an NDVRAVS (SEQ ID NO: 48) sequence.

For example, capsids from Clade F AAV such as AAVhu68 or AAV9 may be selected. Methods of generating vectors having the AAV9 capsid or AAVhu68 capsid, and/or chimeric capsids derived from AAV9 have been described. See, e.g., U.S. Pat. No. 7,906,111, which is incorporated by reference herein. Other AAV serotypes which transduce nasal cells or another suitable target (e.g., muscle or lung) may be selected as sources for capsids of AAV viral vectors including, e.g., AAV1, AAV2, AAV3, AAV4, AAV5, AAV6, AAV6.2, AAV7, AAV8, AAV9, rh10, AAVrh64R1, AAVrh64R2, rh8, AAVrh32.33 (See, e.g., US Published Patent Application No. 2007-0036760-A1; US Published patent application No. 2009-0197338-A1; and EP 1310571). See also, WO 2003/042397 (AAV7 and other simian AAV), U.S. Pat. Nos. 7,790,449 and 7,282,199 (AAV8), WO 2005/033321 (AAV9), and WO 2006/110689, or yet to be discovered, or a recombinant AAV based thereon, may be used as a source for the AAV capsid. See, e.g., WO 2020/223232 A1 (AAV rh90), WO 2020/223231 A1 and International Application No. PCT/US21/45945, filed Aug. 13, 2021 (AAV rh91), and WO 2020/223236 A1 (AAV rh92, AAV rh93, AAV rh9193), which are incorporated herein by reference in its entirety. These documents also describe other AAV which may be selected for generating AAV and are incorporated by reference. In some embodiments, an AAV capsid (cap) for use in the viral vector can be generated by mutagenesis (i.e., by insertions, deletions, or substitutions) of one of the aforementioned AAV caps or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned caps.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(12): 6381-6388, which identifies Clades A, B, C, D, E and F, and provides nucleic acid sequences of novel AAV, GenBank Accession Numbers AY530553 to AY530629. See, also, WO 2005/033321.

As used herein, an “AAV9 capsid” is a self-assembled AAV capsid composed of multiple AAV9 vp proteins. The AAV9 vp proteins are typically expressed as alternative splice variants encoded by a nucleic acid sequence which encodes the vp1 amino acid sequence of GenBank accession: AAS99264. These splice variants result in proteins of different length. In certain embodiments, “AAV9 capsid” includes an AAV having an amino acid sequence which is 99% identical to AAS99264 or 99% identical thereto. See, also, WO 2019/168961, published Sep. 6, 2019, including Table G providing the deamidation pattern for AAV9. See, also U.S. Pat. No. 7,906,111 and WO 2005/033321. As used herein “AAV9 variants” include those described in, e.g., WO2016/049230, U.S. Pat. No. 8,927,514, US 2015/0344911, and U.S. Pat. No. 8,734,809.

A rAAVhu68 is composed of an AAVhu68 capsid and a vector genome. An AAVhu68 capsid is an assembly of a heterogenous population of vp1, a heterogenous population of vp2, and a heterogenous population of vp3 proteins. As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. See, also, PCT/US2018/019992, WO 2018/160582, entitled “Adeno-Associated Virus (AAV) Clade F Vector and Uses Therefor”, and which are incorporated herein by reference in its entirety.

For other recombinant viral vectors, suitable exposed portions of the viral capsid or envelope protein which is responsible for targeting specificity are selected for insertion of the targeting peptide. For example, in an adenovirus, it may be desirable to modify the hexon protein. In a lentivirus, an envelope fusion protein may modified comprise one or more copies of the targeting motif. For vaccinia virus, the major glycoprotein may be modified to comprise one or more copies of the targeting motif. Suitably, these recombinant viral vectors are replication-defective for safety purposes.

### Expression Cassette and Vectors

Vector genomic sequences which are packaged into an AAV capsid and delivered to a host cell are typically composed of, at a minimum, a transgene and its regulatory sequences, and AAV inverted terminal repeats (ITRs). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are about 145 base pairs (bp) in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences are from AAV2. A shortened version of the 5′ ITR, termed ΔITR, has been described in which the D-sequence and terminal resolution site (trs) are deleted. In certain embodiments, the vector genome (e.g., of a plasmid) includes a shortened AAV2 ITR of 130 base pairs, wherein the external A elements is deleted. The shortened ITR may revert back to the wild-type length of 145 base pairs during vector DNA amplification using the internal A element as a template and packaging into the capsid to form the viral particle. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein.

Examples of constitutive promoters suitable for controlling expression of the therapeutic products include, but are not limited to chickenβ-actin (CB) promoter, CB7 promoter, human cytomegalovirus (CMV) promoter, ubiquitin C promoter (UbC), the early and late promoters of simian virus 40 (SV40), U6 promoter, metallothionein promoters, EF1α promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86: 10006-10010 (1989)), the long terminal repeats (LTR) of Moloney Leukemia Virus and other retroviruses, the thymidine kinase promoter of Herpes Simplex Virus and other constitutive promoters known to those of skill in the art. Examples of tissue- or cell-specific promoters suitable for use in the present invention include, but are not limited to, endothelin-I (ET-I) and Flt-I, which are specific for endothelial cells, FoxJ1 (that targets ciliated cells). Other examples of tissue specific promoters suitable for use in the present invention include, but are not limited to, liver-specific promoters. Examples of liver-specific promoters may include, e.g., thyroid hormone-binding globulin (TBG), albumin, Miyatake et al., (1997) J. Virol., 71:5124 32; hepatitis B virus core promoter, Sandig et al., (1996) Gene Ther., 3:1002 9; or human alpha 1-antitrypsin, phosphoenolpyruvate carboxykinase (PECK), or alpha fetoprotein (AFP), Arbuthnot et al., (1996) Hum. Gene Ther., 7:1503 14). Preferably, such promoters are of human origin.

Inducible promoters suitable for controlling expression of the therapeutic product include promoters responsive to exogenous agents (e.g., pharmacological agents) or to physiological cues. These response elements include, but are not limited to a hypoxia response element (HRE) that binds HIF-Iα and β, a metal-ion response element such as described by Mayo et al. (1982, Cell 29:99-108); Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., ppI67-220, 1991).

In one embodiment, expression of the gene product is controlled by a regulatable promoter that provides tight control over the transcription of the sequence encoding the gene product, e.g., a pharmacological agent, or transcription factors activated by a pharmacological agent or in alternative embodiments, physiological cues. Promoter systems that are non-leaky and that can be tightly controlled are preferred.

Examples of regulatable promoters which are ligand-dependent transcription factor complexes that may be used in the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published patent application Nos. 2006/0014711, 2007/0161086, and International Published Application No. WO 01/70816. Examples of chimeric ecdysone receptor systems are described in U.S. Pat. No. 7,091,038, U.S. Published patent application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, MA).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the soluble hACE2 construct can be controlled, for example, by the Tet-on/off system (Gossen et al., 1995, Science 268:1766-9; Gossen et al., 1992, Proc. Natl. Acad. Sci. USA, 89(12):5547-51); the TetR-KRAB system (Urrutia R., 2003, Genome Biol., 4(10):231; Deuschle U et al., 1995, Mol Cell Biol. (4):1907-14); the mifepristone (RU486) regulatable system (Geneswitch; Wang Y et al., 1994, Proc. Natl. Acad. Sci. USA, 91(17):8180-4; Schillinger et al., 2005, Proc. Natl. Acad. Sci. USA. 102(39):13789-94); and the humanized tamoxifen-dep regulatable system (Roscilli et al., 2002, Mol. Ther. 6(5):653-63).

In another aspect, the gene switch is based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof referred to as “rapalogs”. Examples of suitable rapamycins are provided in the documents listed above in connection with the description of the ARGENT™ system. In one embodiment, the molecule is rapamycin [e.g., marketed as Rapamune™ by Pfizer]. In another embodiment, a rapalog known as AP21967 [ARIAD] is used. Examples of these dimerizer molecules that can be used in the present invention include, but are not limited to rapamycin, FK506, FK1012 (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc. Natl. Acad. Sci. USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck]. In certain embodiments, rapamycin or a suitable analog may be delivered locally to the AAV-transfected cells of the nasopharynx. This local delivery may be by intranasal injection, topically to the cells via bolus, cream, or gel. See US patent application US 2019/0216841 A1, which is incorporated herein by reference.

Other suitable enhancers include those that are appropriate for a desired target tissue indication. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. Other suitable introns include those known in the art, e.g., such as are described in WO 2011/126808. Examples of suitable polyadenylation (polyA) sequences include, e.g., rabbit binding globulin (also referenced to as rabbit beta globin, or rBG), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence (see, e.g., MA Zanta-Boussif, et al, Gene Therapy (2009) 16: 605-619).

An AAV viral vector may include multiple transgenes. In certain situations, a different transgene may be used to encode each subunit of a protein (e.g., an immunoglobulin domain, an immunoglobulin heavy chain, an immunoglobulin light chain). In one embodiment, a cell produces the multi-subunit protein following infected/transfection with the virus containing each of the different subunits. In another embodiment, different subunits of a protein may be encoded by the same transgene. An IRES is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., ML Donnelly, et al, (January 1997) J. Gen. Virol., 78 (Pt 1):13-21; S. Furler, S et al, (June 2001) Gene Ther., 8(11):864-873; H. Klump, et al., (May 2001) Gene Ther., 8(10):811-817. This 2A peptide is significantly smaller than IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

In addition to the elements identified above for the expression cassette, the vector also includes conventional control elements which are operably linked to the coding sequence in a manner which permits transcription, translation and/or expression of the encoded product (e.g., soluble hACE2 construct, an anti-influenza antibody, an anti-COVID19 antibody) in a cell transfected with the plasmid vector or infected with the virus produced by the invention. Examples of other suitable transgenes are provided herein. As used herein, “operably linked” sequences include both expression control sequences that are contiguous with the gene of interest and expression control sequences that act in trans or at a distance to control the gene of interest.

Expression control sequences include appropriate enhancer; transcription factor; transcription terminator; promoter; efficient RNA processing signals such as splicing and polyadenylation (polyA) signals; sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. In one embodiment, the regulatory sequences are selected such that the total rAAV vector genome is about 2.0 to about 5.5 kilobases in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. The size of the vector genome may be manipulated based on the size of the regulatory sequences including the promoter, enhancer, intron, poly A, etc. See, Wu et al, Mol. Ther., January 2010 18(1):80-6, which is incorporated herein by reference.

Thus, in one embodiment, an intron is included in the vector. Suitable introns include chicken beta-actin intron, the human beta globin IVS2 (Kelly et al, Nucleic Acids Research, 43(9):4721-32 (2015)); the Promega chimeric intron (Almond, B. and Schenborn, E. T. A Comparison of pCI-neo Vector and pcDNA4/HisMax Vector); and the hFIX intron. Various introns suitable herein are known in the art and include, without limitation, those found at bpg.utoledo.edu/˜afedorov/lab/eid.html, which is incorporated herein by reference. See also, Shepelev V., Fedorov A. Advances in the Exon-Intron Database. Briefings in Bioinformatics 2006, 7: 178-185, which is incorporated herein by reference.

Several different viral genomes were generated in the studies described herein. However, it will be understood by the skilled artisan that other genomic configurations, including other regulatory sequences may be substituted for the promoter, enhancer and other coding sequences may be selected.

rAAV Vector Production

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

In certain embodiments, the inclusion of the at least one copy of the N-x-(T/I/V/A)-(K/R) motif into an AAV capsid provides advantages in production as compared to the method without inclusion of at least one copy of motif in AAV capsid, and wherein the production cells are 293 cells.

Methods of preparing AAV-based vectors (e.g., having an AAV9 or another AAV capsid) are known. See, e.g., US Published patent application No. 2007/0036760 (Feb. 15, 2007), which is incorporated by reference herein. The invention is not limited to the use of AAV9 or other clade F AAV amino acid sequences, but encompasses peptides and/or proteins containing the terminal β-galactose binding generated by other methods known in the art, including, e.g., by chemical synthesis, by other synthetic techniques, or by other methods. The sequences of any of the AAV capsids provided herein can be readily generated using a variety of techniques. Suitable production techniques are well known to those of skill in the art. See, e.g., Sambrook et al, Molecular Cloning: A Laboratory Manual, Cold Spring Harbor Press (Cold Spring Harbor, NY). Alternatively, peptides can also be synthesized by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These methods may involve, e.g., culturing a host cell which contains a nucleic acid sequence encoding an AAV capsid; a functional rep gene; a minigene composed of, at a minimum, AAV inverted terminal repeats (ITRs) and a transgene; and sufficient helper functions to permit packaging of the minigene into the AAV capsid protein. These and other suitable production methods are within the knowledge of those of skill in the art and are not a limitation of the present invention.

The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

These rAAVs are particularly well suited to gene delivery for therapeutic purposes and for preventing infection. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art. Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant AAV described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, cells are manufactured in a suitable cell culture (e.g., HEK 293 cells). Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest for packaging into the capsid, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. In certain embodiments, the methods of making and using AAV production systems includes that of which uses pseudorabies viruses (rPRV) described in U.S. patent application 63/016,894 filed on Apr. 28, 2020, incorporated herein by reference.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International patent application No. PCT/US2016/065970, filed Dec. 9, 2016, entitled “Scalable Purification Method for AAV9”, which is incorporated by reference. Purification methods for AAV8, International patent application No. PCT/US2016/065976, filed Dec. 9, 2016, and rh10, International patent application No. PCT/US16/66013, filed Dec. 9, 2016, entitled “Scalable Purification Method for AAVrh10”, also filed Dec. 11, 2015, and for AAV1, International patent application No. PCT/US2016/065974, filed Dec. 9, 2016 for “Scalable Purification Method for AAV1”, filed Dec. 11, 2015, are all incorporated by reference herein. To calculate empty and full particle content, vp3 band volumes for a selected sample (e.g., in examples herein an iodixanol gradient-purified preparation where number of GC=number of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; and Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

Additionally, another example of measuring empty to full particle ratio is also known in the art. Sedimentation velocity, as measured in an analytical ultracentrifuge (AUC) can detect aggregates, other minor components as well as providing good quantitation of relative amounts of different particle species based upon their different sedimentation coefficients. This is an absolute method based on fundamental units of length and time, requiring no standard molecules as references. Vector samples are loaded into cells with 2-channel charcoal-epon centerpieces with 12 mm optical path length. The supplied dilution buffer is loaded into the reference channel of each cell. The loaded cells are then placed into an AN-60Ti analytical rotor and loaded into a Beckman-Coulter ProteomeLab XL-I analytical ultracentrifuge equipped with both absorbance and RI detectors. After full temperature equilibration at 20° C. the rotor is brought to the final run speed of 12,000 rpm. A₂₈₀ scans are recorded approximately every 3 minutes for ˜5.5 hours (110 total scans for each sample). The raw data is analyzed using the c(s) method and implemented in the analysis program SEDFIT. The resultant size distributions are graphed and the peaks integrated. The percentage values associated with each peak represent the peak area fraction of the total area under all peaks and are based upon the raw data generated at 280 nm; many labs use these values to calculate empty: full particle ratios. However, because empty and full particles have different extinction coefficients at this wavelength, the raw data can be adjusted accordingly. The ratio of the empty particle and full monomer peak values both before and after extinction coefficient-adjustment is used to determine the empty-full particle ratio.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay. Quantification also can be done using ViroCyt or flow cytometry.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum. Gene Ther. Methods. 2014 April; 25(2):115-25. doi: 10.1089/hgtb.2013.131. Epub 2014 Feb. 14.

### Therapeutic Proteins and Delivery Systems

Fusion partners, conjugate partners and recombinant vectors containing the targeting motif provided herein, N-x-(T/I/V/A)-(K/R) motif, are useful with a variety of different therapeutic proteins, polypeptides, nanoparticles, and delivery systems. Examples of proteins and compounds useful in compositions provided herein and targeted delivery include the following. It will be understood that the viral vectors, nanoparticles and other delivery systems contain sequences encoding the selected proteins (or conjugates) for expression in vivo.

In certain embodiments, the protein is MCT8 protein (SLC16A2 gene) and other compounds for treating of Allan-Herndon-Dudley disease and the symptoms thereof.

In certain embodiments, the protein is selected from a disease associated with a transport defect such as, e.g., cystic fibrosis (a cystic fibrosis transmembrane regulator), alpha-1-antitrypsin (hereditary emphysema), FE (hereditary hemochromatosis), tyrosinase (oculocutaneous albinism), Protein C (protein C deficiency), Complement C inhibitor (type I hereditary angioedema), alpha-D-galactosidase (Fabry disease), beta hexosaminidase (Tay-Sachs), sucrase-isomaltase (congenital sucrase-isomaltase deficiency), UDP-glucoronosyl-transferase (Crigler-Najjar type II), insulin receptor (diabetes mellitus), growth hormone receptor (laron syndrome), among others. Examples of other genes and proteins those associated with, e.g. spinal muscular atrophy (SMA, SMN1), Huntingdon's Disease, Rett Syndrome (e.g., methyl-CpG-binding protein 2 (MeCP2); UniProtKB—P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), among others. See, e.g., www.orpha.net/consor/cgi-bin/Disease_Search_List.php; rarediseases.info.nih.gov/diseases. Further illustrative genes which may be delivered via the rAAV include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; methylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease.

Examples of proteins and compounds useful in compositions provided herein and targeted delivery include therapeutic proteins and other compounds and vaccine protein derivatives of the following respiratory-associated infectious diseases and passive immunoglobulins direct against these infectious disease. Examples of suitable therapeutic proteins include, e.g., alpha-1-antitrypsin, cystic fibrosis transmembrane protein, and variants thereof, surfactant-B, bone morphogenetic protein receptor type II (associated with pulmonary arterial hypertension), and various cancer therapeutics.

Examples of suitable vaccine or passive immunization include proteins derived from airborne pathogens, including the human respiratory coronaviruses, have been associated with severe acute respiratory syndrome (SARS-CoV1), the common cold, and non A, B or C hepatitis. SARS-CoV2 is the causative agent of COVID-19 and antibodies specific for this virus have been described. Examples of IgG antibodies which have been described as being useful for binding the spike protein of human ACE2 of SARS-CoV2 and having neutralizing activity include, e.g., LY-CoV555 (Eli Lilly), TY027 (Tychon), STI-1499 and STI-2020 (COVI-GUARD; Sorrento), 80R, AD1055689/56046 (Adimab) (Renn et al., Trends in Pharmacological Sciences, 2020); BD-217, BD-218, BD-236 (Cao et al., Cell, 182, 73-84 (2020)). Examples of IgG antibodies which have been described as being useful for binding the receptor binding domain (RBD) of human ACE2 of SARS-COV2 and having neutralizing activity include, e.g., COV2-2196, COV2-2130, COV2-2165 (Zost et al., Nature, 584, 443-465 (2020)); BD-361, BD-368, BD-368-2 (Cao et al., Cell, 182, 73-84 (2020)); B38, H4 (Y. Wu et al., Science 10.1126/science.abc2241 (2020); Jahanshahlu and Rezaei, Biomedicine and Pharmacotherapy 129 (2020)); S309, S315, S304 (Pinto et al., Nature, 583, 290-311 (2020)); CC6.29, CC6.30, CC6.33, CC12.1, CC12.3 (Rogers et al., Science 369, 956-963 (2020)); JS016 (Eli Lilly), CA1, CB6-LALA, P2C-1F11/P2B-2F6/P2A-1A3, 311mab-31B5311/32D4, COVA 2-15, 414-1, (Renn et al., Trends in Pharmacological Sciences, 2020). Examples of IgG antibodies which have been described as being useful for binding the spike protein of human ACE2 of SARS-COV1 and having neutralizing activity include, e.g., m396 and CR3104 (Prabakaran et al., Journal of Biological Chemistry, 281, 15829-15836 (2006); ter Meulen et al., PLoS, 3, 7 (2006)). Examples of IgG antibodies which have been described as being useful for binding either RBD or spike protein of human ACE2 of both SARS-COV1 and SARS-CoV2 and having neutralizing activity include, e.g., CR3022 and 47D11 (Wang et al., Nature Communications, 11, nature.com/naturecommunications (2020)).

Examples of other target viruses include influenza virus from the orthomyxovirudae family, which includes: Influenza A, Influenza B, and Influenza C. The type A viruses are the most virulent human pathogens. The serotypes of influenza A which have been associated with pandemics include, H1N1, which caused Spanish Flu in 1918, and Swine Flu in 2009; H2N2, which caused Asian Flu in 1957; H3N2, which caused Hong Kong Flu in 1968; H5N1, which caused Bird Flu in 2004; H7N7; H1N2; H9N2; H7N2; H7N3; and H10N7. Broadly neutralizing antibodies against influenza A have been described. As used herein, a “broadly neutralizing antibody” refers to a neutralizing antibody which can neutralize multiple strains from multiple subtypes. For example, CR6261 [The Scripps Institute/Crucell] has been described as a monoclonal antibody that binds to a broad range of the influenza virus including the 1918 “Spanish flu” (SC1918/H1) and to a virus of the H5N1 class of avian influenza that jumped from chickens to a human in Vietnam in 2004 (Viet04/H5). CR6261 recognizes a highly conserved helical region in the membrane-proximal stem of hemagglutinin, the predominant protein on the surface of the influenza virus. This antibody is described in WO 2010/130636, incorporated by reference herein. Another neutralizing antibody, F10 [XOMA Ltd] has been described as being useful against H1N1 and H5N1. [Sui et al, Nature Structural and Molecular Biology (Sui, et al. 2009, 16(3):265-73)] Other antibodies against influenza, e.g., Fab28 and Fab49, may be selected. See, e.g., WO 2010/140114 and WO 2009/115972, which are incorporated by reference. Still other antibodies, such as those described in WO 2010/010466, US Published patenttent Publication US/2011/076265, and WO 2008/156763, may be readily selected.

Other target pathogenic viruses include, arenaviruses (including funin, machupo, and Lassa), filoviruses (including Marburg and Ebola), hantaviruses, picornaviridae (including rhinoviruses, echovirus), coronaviruses, paramyxovirus, morbillivirus, respiratory syncytial virus, togavirus, coxsackievirus, parvovirus B19, parainfluenza, adenoviruses, reoviruses, variola (Variola major (Smallpox)) and Vaccinia (Cowpox) from the poxvirus family, and varicella-zoster (pseudorabies). Viral hemorrhagic fevers are caused by members of the arenavirus family (Lassa fever) (which family is also associated with Lymphocytic choriomeningitis (LCM)), filovirus (ebola virus), and hantavirus (puremala). The members of picornavirus (a subfamily of rhinoviruses), are associated with the common cold in humans. The coronavirus family, which includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog). The paramyxovirus family includes parainfluenza Virus Type 1, parainfluenza Virus Type 3, bovine parainfluenza Virus Type 3, rubelavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus (RSV). The parvovirus family includes feline parvovirus (feline enteritis), feline panleucopeniavirus, canine parvovirus, and porcine parvovirus. The adenovirus family includes viruses (EX, AD7, ARD, O.B.) which cause respiratory disease.

A neutralizing antibody construct against a bacterial pathogen may also be selected for use in the present invention. In one embodiment, the neutralizing antibody construct is directed against the bacteria itself. In another embodiment, the neutralizing antibody construct is directed against a toxin produced by the bacteria. Examples of airborne bacterial pathogens include, e.g., Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), Legionella pneumonia (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria (diphtheria), Mycoplasma pneumoniae (pneumonia). The causative agent of anthrax is a toxin produced by Bacillus anthracis. Neutralizing antibodies against protective agent (PA), one of the three peptides which form the toxoid, have been described. The other two polypeptides consist of lethal factor (LF) and edema factor (EF). Anti-PA neutralizing antibodies have been described as being effective in passively immunization against anthrax. See, e.g., U.S. Pat. No. 7,442,373; R. Sawada-Hirai et al, J Immune Based Ther Vaccines. 2004; 2: 5. (on-line 2004 May 12). Still other anti-anthrax toxin neutralizing antibodies have been described and/or may be generated. Similarly, neutralizing antibodies against other bacteria and/or bacterial toxins may be used to generate a non-IgG antibody as described herein.

Other infectious diseases may be caused by airborne fungi including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolyspora faeni, Thermoactinomyces vulgaris, Alternaria alternate, Cladosporium species, Helminthosporium, and Stachybotrys species.

In addition to airborne infectious disease conditions which affect humans, many of which are described above, passive immunization according to the invention may be used to prevent conditions associated with direct inoculation of the nasal passages, e.g., conditions which may be transmitted by direct contact of the fingers with the nasal passages. These conditions may include fungal infections (e.g., athlete's foot), ringworm, or viruses, bacteria, parasites, fungi, and other pathogens which can be transmitted by direct contact. In addition, a variety of conditions which affect household pets, cattle and other livestock, and other animals. For example, in dogs, infection of the upper respiratory tract by canine sinonasal aspergillosis causes significant disease. In cats, upper respiratory disease or feline respiratory disease complex originating in the nose causes morbidity and mortality if left untreated. Cattle are prone to infections by the infectious bovine rhinotracheitis (commonly called IBR or red nose) is an acute, contagious virus disease of cattle. In addition, cattle are prone to Bovine Respiratory Syncytial Virus (BRSV) which causes mild to severe respiratory disease and can impair resistance to other diseases. Still other pathogens and diseases will be apparent to one of skill in the art.

An antibody, and particularly, a neutralizing antibody, against a pathogen such as those specifically identified herein (e.g., anti-SARS-CoV2, anti-SARS-CoV1, anti-influenza, anti-ebola, anti-RSV), may be used to generate a class-switched or non-IgG antibody. Monoclonal antibodies (mAbs) with broad neutralizing capacity can be identified using antibody phage display to screen libraries from donors recently vaccinated with the seasonal flu vaccine, from non-immune humans or from survivors of a natural infection. In the case of influenza, antibodies have been identified which neutralize more than one influenza subtype by blocking viral fusion with the host cell. This technique may be utilized with other infections to obtain a neutralizing monoclonal antibody. See, e.g., U.S. Pat. No. 5,811,524, which describes generation of anti-respiratory syncytial virus (RSV) neutralizing antibodies. The techniques described therein are applicable to other pathogens. Such an antibody may be used intact, or its sequences (scaffold) modified to generate an artificial or recombinant neutralizing antibody construct. Such methods have been described [see, e.g., WO 2010/13036; WO 2009/115972; WO 2010/140114]. In one embodiment, mouse, rat, hamster or other host animals, is immunized with an immunizing agent to generate lymphocytes that produce antibodies with binding specificity to the immunizing antigen. In an alternative approach, the lymphocytes may be immunized in vitro. Human antibodies can be produced using techniques such as phage display libraries (Hoogenboom and Winter, J. Mol. Biol, 1991, 227:381, Marks et al., J. Mol. Biol. 1991, 222:581).

### Compositions and Uses

Provided herein are compositions containing at least one rAAV stock (e.g., an rAAV9 or rAAVhu68 mutant stock) and an optional carrier, excipient and/or preservative. An rAAV stock refers to a plurality of rAAV vectors which are the same, e.g., such as in the amounts described below in the discussion of concentrations and dosage units.

In certain embodiments, a composition may contain at least a second, different rAAV stock. This second vector stock may vary from the first by having a different AAV capsid and/or a different vector genome. In certain embodiments, a composition as described herein may contain a different vector expressing an expression cassette as described herein, or another active component (e.g., an antibody construct, another biologic, and/or a small molecule drug).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among nonionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one embodiment, the formulation buffer is phosphate-buffered saline (PBS) with total salt concentration of 200 mM, 0.001% (w/v) pluronic F68 (Final Formulation Buffer, FFB).

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. In certain embodiments, the vectors are formulated for delivery via intranasal delivery devices for targeted delivery to nasal and/or nasopharynx epithelial cells. In certain embodiments, vectors are formulated for aerosol delivery devices, e.g., via a nebulizer or through other suitable devices. Other conventional and pharmaceutically acceptable routes of administration include, but are not limited to, direct delivery to a desired organ (e.g., lung), oral inhalation, intrathecal, intratracheal, intraarterial, intraocular, intravenous, intramuscular, subcutaneous, intradermal, and other parenteral routes of administration. In one embodiment, the vector is administered intranasally using intranasal mucosal atomization device (LMA® MAD Nasal™-MAD110). In another embodiment the vector is administered intrapulmonary in nebulized form using Vibrating Mesh Nebulizer (Aerogen® Solo) or MADgic™ Laryngeal Mucosal Atomizer. Routes of administration may be combined, if desired. Routes of administration and utilization of which for delivering rAAV vectors are also described in the following published US patent applications, the contents of each of which is incorporated herein by reference in its entirety: US 2018/0155412A1, US 2018/0243416A1, US 2014/0031418 A1, and US 2019/0216841A1.

Dosages of the viral vector will depend primarily on factors such as the condition being treated, the age, weight and health of the patient, and may thus vary among patients. For example, a therapeutically effective human dosage of the viral vector is generally in the range of from about 25 to about 1000 microliters to about 5 mL of aqueous suspending liquid containing doses of from about 10⁹ to 4×10¹⁴ GC of AAV vector. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 10⁹ GC to about 10¹⁶ GC (to treat an average subject of 70 kg in body weight) including all integers or fractional amounts within the range, and preferably 10¹² GC to 10¹⁴ GC for a human patient. In one embodiment, the compositions are formulated to contain at least 10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 10¹⁰ to about 10¹² GC per dose including all integers or fractional amounts within the range. In one embodiment, for human application the dose can range from 10⁹ to about 7×10¹³ GC per dose including all integers or fractional amounts within the range. In one embodiment, for human application the dose ranges from 6.25×10¹² GC to 5.00×10¹³ GC. In a further embodiment, the dose is about 6.25×10¹² GC, about 1.25×10¹³ GC, about 2.50×10¹³ GC, or about 5.00×10¹³ GC. In certain embodiment, the dose is divided into one half thereof equally and administered to each nostril. In certain embodiments, for human application the dose ranges from 6.25×10¹² GC to 5.00×10¹³ GC administered as two aliquots of 0.2 ml per nostril for a total volume delivered in each subject of 0.8 ml.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In certain embodiments, the recombinant vectors may be dosed intranasally by using two sprays to each nostril. In one embodiment, the two sprays are administered by alternating to each nostril, e.g., left nostril spray, right nostril spray, then left nostril spray, right nostril spray. In certain embodiments, there may be a delay between alternating sprays. For example, each nostril may receive multiple sprays which are separated by an interval of about 10 to 60 seconds, or 20 to 40 seconds, or about 30 seconds, to a few minutes, or longer. Such sprays may deliver, e.g., about 150 μL to 300 μL, or about 250 μL in each spray, to achieve a total volume dosed of about 200 μL to about 600 μL, 400 μL to 700 μL, or 450 μL to 1000 μL.

In certain embodiment, the recombinant AAV vector may be dosed intranasally to achieve a concentration of 5-20 ng/ml of the expression product of the transgene as measured in a nasal wash solution post-dosing, e.g., one week to four weeks, or about two weeks after administration of the vector. Methods of acquiring the nasal wash solution in the subjected as well as methods of quantification of the expression product of the transgene are conventional.

For other routes of administration, e.g., intravenous or intramuscular, dose levels would be higher than for intranasal delivery. For example, such suspensions may be volumes doses of about 1 mL to about 25 mL, with doses of up to about 2.5×10⁵ GC.

In certain embodiments, the intranasal delivery device provides a spay atomizer which delivers a mist of particles having an average size range of about 30 microns to about 100 microns in size. In certain embodiments, the average size range is about 10 microns to about 50 microns. Suitable devices have been described in the literature and some are commercially available, e.g., the LMA MAD NASAL™ (Teleflex Medical; Ireland); Teleflex VaxINator™ (Teleflex Medical; Ireland); Controlled Particle Dispersion® (CPD) from Kurve Technologies. See, also, P G Djupesland, Drug Deliv and Transl. Res (2013) 3: 42-62. In certain embodiments, the particle size and volume of delivery is controlled in order to preferentially target nasal epithelial cells and minimize targeting to the lung. In other embodiments, the mist of particles is about 0.1 micron to about 20 microns, or less, in order to deliver to lung cells. Such smaller particle sizes may minimize retention in the nasal epithelium.

One device mists particles at an average diameter of about 16 microns to about 22 microns. The mist may be delivered directly to the tracheobronchial tree inserted through the suction channel of a 3.5-mm flexible fiberoptic bronchoscope (Olympus, Melville, NY). Other suitable delivery devices may include a laryngo-tracheal mucosal atomizer, which provides for administration across the upper airway past the vocal cords. It fits through vocal cords and down a laryngeal mask or into nasal cavity. The droplets are atomized at an average diameter of about 30 microns to about 100 microns. A standard device has a tip diameter of about 0.18 in (4.6 mm), a length of about 4.5-8.5 inches, and is inserted through the suction channel and advanced approximately 3 mm beyond the distal tip of the scope. Doses may be administered is 10 aliquots (approximately 150 μl each) of control with saline or rAAV sprayed into right and left main stem bronchi.

In one embodiment, a frozen composition is provided which contains an rAAV in a buffer solution as described herein, in frozen form. Optionally, one or more surfactants (e.g., Pluronic F68), stabilizers or preservatives is present in this composition. Suitably, for use, a composition is thawed and titrated to the desired dose with a suitable diluent, e.g., sterile saline or a buffered saline.

In one embodiment, a composition comprising one or more exogenous endothelial cell targeting peptide from the motif: N-x-(T/I/V/A)-(K/R) (SEQ ID NO: 47) and optional flanking linker sequences are provided, together with one or more of a physiologically compatible carrier, excipient, and/or aqueous suspension base. Further provided are compositions comprising nucleic acid sequences encoding same. In certain embodiments, the targeting peptide is of SEQ ID NO: 40 and is encoded by a nucleic acid sequence of SEQ ID NO: 54, or a sequence at least about 70% identical thereto. In certain embodiments, the targeting peptide is of SEQ ID NO: 38 and is encoded by a nucleic acid sequence of SEQ ID NO: 50, or a sequence at least about 70% identical thereto. In certain embodiments, the targeting peptide is of SEQ ID NO: 46 and is encoded by a nucleic acid sequence of SEQ ID NO: 56, or a sequence at least about 70% identical thereto. In certain embodiments, the targeting peptide is of SEQ ID NO: 43 and is encoded by a nucleic acid sequence of SEQ ID NO: 52, or a sequence at least about 70% identical thereto. In certain embodiments, the targeting peptide is of SEQ ID NO: 39 and is encoded by a nucleic acid sequence of SEQ ID NO: 55, or a sequence at least about 70% identical thereto. In certain embodiments, the targeting peptide is of SEQ ID NO: 42 and is encoded by a nucleic acid sequence of SEQ ID NO: 51, or a sequence at least about 70% identical thereto. In certain embodiments, the targeting peptide is of SEQ ID NO: 41 and is encoded by a nucleic acid sequence of SEQ ID NO: 53, or a sequence at least about 70% identical thereto.

In another embodiment, a fusion polypeptide or protein is provided comprising one or more exogenous brain endothelial cell targeting peptide from the motif: N-x-(T/I/V/A)-(K/R) (SEQ ID NO: 47) are provided and fusion partner which comprises at least one polypeptide or protein. Further provided are nucleic acid sequences encoding same.

In certain embodiments, a composition comprising a fusion polypeptide or protein, or a nucleic acid sequence encoding the fusion polypeptide or protein, or a nanoparticle containing same are provided. The composition may further comprise one or more of a physiologically compatible carrier, excipient, and/or aqueous suspension base.

In certain embodiments, a nucleic acid sequence encoding the fusion polypeptide protein is encapsulated in a lipid nanoparticle (LNP). As used herein, the phrase “lipid nanoparticle” or “nanoparticle” refers to a transfer vehicle comprising one or more lipids (e.g., cationic lipids, non-cationic lipids, and PEG-modified lipids). Preferably, the lipid nanoparticles are formulated to deliver one or more nucleic acid sequences to one or more target cells (e.g., liver and/or muscle). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkylcyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a nucleic acid sequence encapsulated therein to a target cell. Useful lipid nanoparticles for nucleic acid sequence comprise a cationic lipid to encapsulate and/or enhance the delivery of such nucleic acid sequence into the target cell that will act as a depot for protein production. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid, PEG-lipid and polymer forming a lipid bilayer around encapsulated nucleic acid sequence (Kowalski et al., 2019, Mol. Ther. 27(4):710-728). In some embodiments, LNP comprises a cationic lipids (i.e. N-[1-(2,3-dioleoyloxy)propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino)esters (PBAEs). See, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference.

In certain embodiments, a composition, e.g., an rAAV having a modified capsid with a N-x-(T/I/V/A)-(K/R) (SEQ ID NO: 47) peptide and optional linker sequences, a fusion polypeptide or protein, or a conjugate comprising a nanoparticle or chemical moiety, is useful for delivering a therapeutic to a patient in need thereof. In certain embodiments, the method is for targeting therapy to the brain endothelial cells. In certain embodiments, the method is for treating Allan-Herndon-Dudley disease by delivering an MCT8 protein (e.g., UniProt ID No.: P36021) or a gene which expresses MCT8 in vivo. In other embodiments, the method is for targeting therapy to the lung. In certain embodiments, the delivered product is a soluble Ace2 protein (e.g., hAce2 decoy or hAce2 decoy fusion), an anti-SARS antibody, an anti-SARS-CoV2 antibody, an anti-influenza antibody, or a cystic fibrosis transmembrane protein. See also, omim.org/entry/300523, the content of which is incorporated herein by reference. See also, U.S. Provisional Application No. 63/143,614, filed Jan. 29, 2021, US Provisional Application No. 63/1650,511, filed Mar. 12, 2021, and U.S. patent application No. 63/166,686, filed Mar. 26, 2021, U.S. Provisional patent application No. 63/215,159, filed Jun. 25, 2021, U.S. Provisional Application No. 63/253,654, filed Oct. 8, 2021 are incorporated herein by reference.

In certain embodiments, a rAAV having a modified capsid as described herein may be delivered in a co-therapeutic regimen which further comprises one or more other active components. In certain embodiments, the regimen may involve co-administration of an immunomodulatory component. Such an immunomodulatory regimen may include, e.g., but are not limited to immunosuppressants such as, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25−) or CD3-directed antibodies, anti-IL-2 antibodies, cyclosporin, tacrolimus, sirolimus, IFN-β, IFN-γ, an opioid, or TNF-α (tumor necrosis factor-alpha) binding agent. In certain embodiments, the immunosuppressive therapy may be started prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week, about 15 days, about 30 days, about 45 days, 60 days, or longer, as needed. Still other co-therapeutics may include, e.g., anti-IgG enzymes, which have been described as being useful for depleting anti-AAV antibodies (and thus may permit administration to patients testing above a threshold level of antibody for the selected AAV capsid), and/or delivery of anti-FcRN antibodies which is described, e.g., in U.S. Provisional patent application No. 63/040,381, filed Jun. 17, 2020, entitled “Compositions and Methods for Treatment of Gene Therapy Patients”, and/or one or more of a) a steroid or combination of steroids and/or (b) an IgG-cleaving enzyme, (c) an inhibitor of Fc-IgE binding; (d) an inhibitor of Fc-IgM binding; (e) an inhibitor of Fc-IgA binding; and/or (f) gamma interferon.

An antibody “Fc region” refers to the crystallizable fragment which is the region of an antibody which interacts with the cell surface receptors (Fc receptors). In one embodiment, the Fc region is a human IgG1 Fc. In one embodiment, the Fc region is a human IgG2 Fc. In one embodiment, the Fc region is a human IgG4 Fc. In one embodiment, the Fc region is an engineered Fc fragment. See, e.g., Lobner, Elisabeth, et al. “Engineered IgG1-Fc-one fragment to bind them all.” Immunological reviews 270.1 (2016): 113-131; Saxena, Abhishek, and Donghui Wu. “Advances in therapeutic Fc engineering-modulation of IgG-Associated effector functions and serum half-life.” Frontiers in immunology 7 (2016); Irani, Vashti, et al. “Molecular properties of human IgG subclasses and their implications for designing therapeutic monoclonal antibodies against infectious diseases.” Molecular immunology 67.2 (2015): 171-182; Rath, Timo, et al. “Fc-fusion proteins and FcRn: structural insights for longer-lasting and more effective therapeutics.” Critical reviews in biotechnology 35.2 (2015): 235-254; and Invivogen, IgG-Fc Engineering For Therapeutic Use, invivogen.com/docs/Insight200605.pdf, April 2006; each of which is incorporated by reference herein.

An antibody “hinge region” is a flexible amino acid portion of the heavy chains of IgG and IgA immunoglobulin classes, which links these two chains by disulfide bonds.

An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

“Neutralizing antibody titer” (NAb titer) a measurement of how much neutralizing antibody (e.g., anti-AAV NAb) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009, 199 (3): p. 381-390, which is incorporated by reference herein.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine—glycine pairs. Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, 97%, 99%, up to about 100% deamidated at a referenced amino acid position, as compared to the predicted amino acid sequence at the reference amino acid position. Such percentages may be determined using 2D-gel, mass spectrometry techniques, or other suitable techniques.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected. See, e.g., WO 2019/168961, published Sep. 6, 2019, including Table G providing the deamidation pattern for AAV9 and WO 2020/160582, filed Sep. 7, 2018. See, also, e.g., WO 2020/223231, published Nov. 5, 2020 (rh91, including table with deamidation pattern), U.S. Provisional Patent Application No. 63/065,616, filed Aug. 14, 2020, and U.S. Provisional patent application No. 63/109,734, filed Nov. 4, 2020, and International patent application No. PCT/US21/45945, filed Aug. 13, 2021, which are all incorporated herein by reference in its entirety.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

A “recombinant AAV” or “rAAV” is a DNAse-resistant viral particle containing two elements, an AAV capsid and a vector genome containing at least non-AAV coding sequences packaged within the AAV capsid. In certain embodiments, the capsid contains about 60 proteins composed of vp1 proteins, vp2 proteins, and vp3 proteins, which self-assemble to form the capsid. Unless otherwise specified, “recombinant AAV” or “rAAV” may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

The term “nuclease-resistant” indicates that the AAV capsid has assembled around the expression cassette which is designed to deliver a transgene to a host cell and protects these packaged genomic sequences from degradation (digestion) during nuclease incubation steps designed to remove contaminating nucleic acids which may be present from the production process.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s) (i.e., transgene(s)), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding protein of interest operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

As used herein, “operably linked” sequences include both expression control sequences that are contiguous with the gene of interest and expression control sequences that act in trans or at a distance to control the gene of interest.

In certain embodiments, non-viral genetic elements used in manufacture of a rAAV, will be referred to as vectors (e.g., production vectors). In certain embodiments, these vectors are plasmids, but the use of other suitable genetic elements is contemplated. Such production plasmids may encode sequences expressed during rAAV production, e.g., AAV capsid or rep proteins required for production of a rAAV, which are not packaged into the rAAV. Alternatively, such a production plasmid may carry the vector genome which is packaged into the rAAV.

As used herein, a “parental capsid” refers to a non-mutated or a non-modified capsid selected from parvovirus or other viruses (e.g., AAV, adenovirus, HSV, RSV, etc.). In certain embodiments, the parental capsid includes any naturally occurring AAV capsids comprising a wild-type genome encoding for capsid proteins (i.e., vp proteins), wherein the capsid proteins direct the AAV transduction and/or tissue-specific tropism. In some embodiments, the parent capsid is selected from AAV which natively targets CNS. In other embodiments, the parental capsid is selected from AAV which do not natively target CNS.

As used herein, a “variant capsid” or a “variant AAV” or “variant AAV capsid” refers to a modified capsid or a mutated capsid, wherein the capsid protein comprises an insertion of a tissue-specific targeting peptide.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

The term “translation” in the context of the present invention relates to a process at the ribosome, wherein an mRNA strand controls the assembly of an amino acid sequence to generate a protein or a peptide.

The term “expression” is used herein in its broadest meaning and comprises the production of RNA or of RNA and protein. Expression may be transient or may be stable.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., an immunoglobulin region or domain, an AAV cap protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

An effective amount may be determined based on an animal model, rather than a human patient.

As described above, the term “about” when used to modify a numerical value means a variation of ±10%, (±10%, e.g., ±1, ±2, ±3, ±4, ±5, ±6, ±7, ±8, ±9, ±10, or values therebetween) from the reference given, unless otherwise specified.

In certain instances, the term “E+#” or the term “e+#” is used to reference an exponent. For example, “5 E10” or “5 e10” is 5×10¹⁰. These terms may be used interchangeably.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are illustrative only and are not a limitation on the invention described herein.

### Example 1. Primary Screen

It has been shown that small peptide insertions into flexible loop on the surface of the AAV capsid can mediate interactions with new cellular receptors. In one case discovered at CalTech (AAV9-PHP.B), a seven amino acid peptide inserted into the HVR8 loop on AAV9 mediates interaction with Ly6a, a GPI-anchored receptor on the brain vasculature of some mouse strains. This interaction drives transport of AAV9-PHP.B across the blood-brain barrier (BBB), resulting in ˜50-fold higher transduction of brain cells than AAV9. In this work, we search for peptide inserts that can bind cell membrane targets on the BBB and thus have the potential to drive the AAV9 capsid across the BBB.

We sought to solve the AAV-BBB problem by first surveying the available academic and patent literature for peptide sequences that may have the potential to interact with the vascular cells in the brain. We found the following sources of these peptides:
Published results of phage-display experiments in which phage display libraries were panned against primary brain endothelial cells;
Natural ligand peptides to known BBB-resident membrane proteins;
CDRs of antibodies targeted to BBB-resident membrane proteins;
Viral coat proteins of flaviviruses that cause encephalitis; and
Bacterial toxins that have cell-binding activities directed at GPI anchorages.

We generated a library of AAV9 insertion mutants containing hundreds of peptides from these sources, all inserted individually at the HVR8 locus (between position 588 and 589, based on the numbering of the amino acid sequence of AAV9 capsid of SEQ ID NO: 44). Each peptide was typically present in the library in multiple forms that differed by 1) length of peptide inserted 2) presence of flexible GSG or GG linker sequences on both sides of the peptide. Peptides were also encoded using multiple synonymous codons so that we could independently observe replicate activities in the screen.

Additionally, we generated a library of insert variants in HVR8 of AAVhu68 capsid with either known or suspected ligand peptides which target the blood-brain barrier (BBB) receptors (between position 588 and 589, based on the numbering of the amino acid sequence of AAVhu68 capsid of SEQ ID NO: 45). Such were:
Peptides binding mammalian brain endothelium (published phage display data);
Classical RMT receptor ligands (e.g., Tf);
CDRs of mAbs against RMT receptors (e.g., anti-TfR); and
Coat proteins of flaviviruses which cause encephalitis.

As a control a PHP.B peptide was included as well (positive control for C57/BL6 and negative control for Balb/c & NHP). Each peptide was encoded in multiple ways (with and without a linker, and in several synonymous DNA sequences).

We injected this library at high-dose intravenously (IV) to 2 mouse strains and to one non-human primate. After a 2-3 week in-life period, the animals were necropsied, and tissues were collected. We extracted the DNA genomes of AAV vectors from CNS and other tissues, and subjected these to next-generation sequencing (NGS). The vector variants encapsidate their own capsid gene variant, allowing us to track capsid activity through the relative abundance of the capsid gene variant in the tissue of interest. We scored the BBB activity (“enrichment score”) of each variant in the library by calculating its abundance in the CNS normalized to its abundance in the injected library mixture.

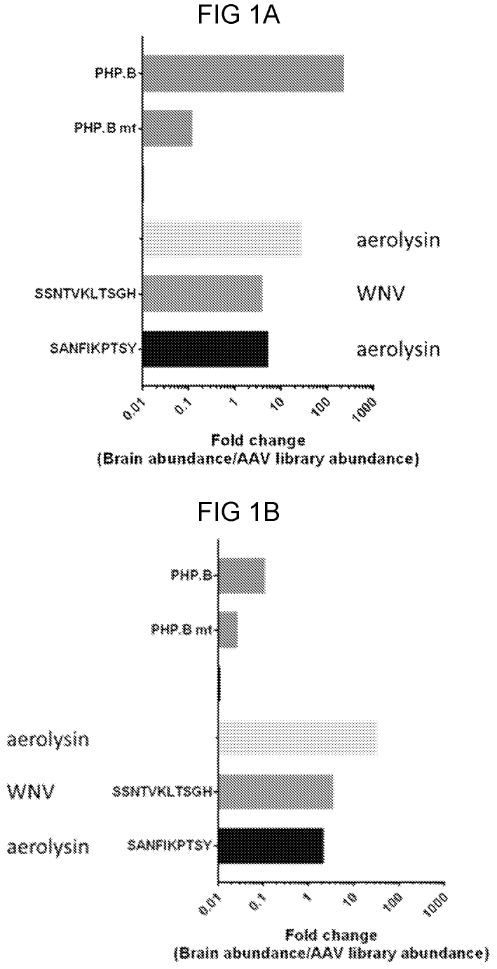

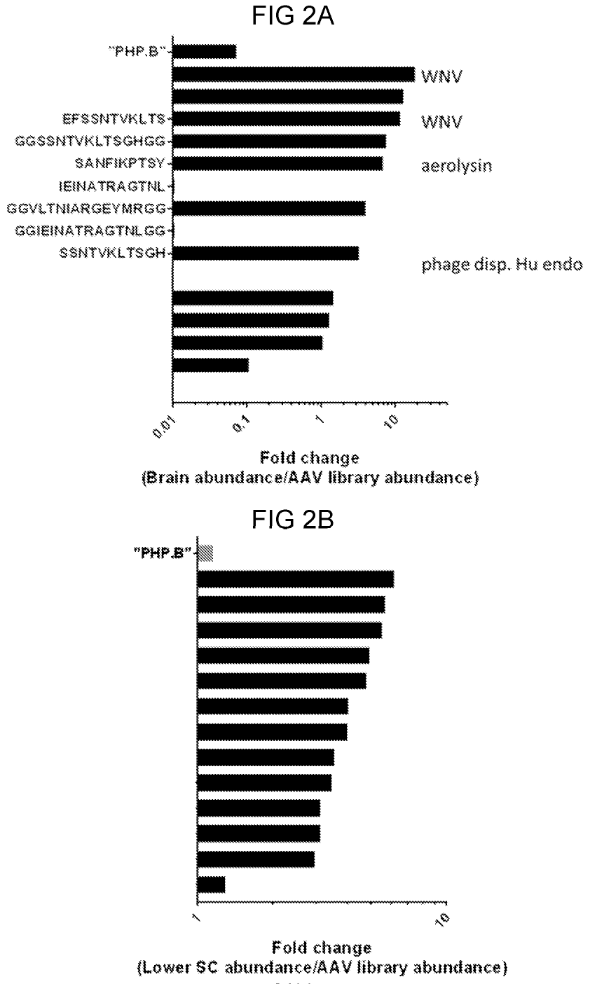

In mouse study, tope brain enriched HVR8 insertions in C57/BL6 mice were: TLAVPFK (SEQ ID NO: 49) (PHP.B), Positive control PHP.B comes up 3 times independently as the most enriched hit. Three of the PHP.B peptides with synonymous codons are independently enriched. Several other peptides are also enriched in brain. FIGS. 1A and 1B show the enrichment scores for the best mouse brain hits in the screen, with reference peptides (FIG. 1A for C57BL/6J mice; and FIG. 1B for Balb/c mice. FIGS. 2A and 2B show the enrichment scores for the top performing NHP brain (FIG. 2A) and spinal cord (FIG. 2B) tissue.

TABLE 1
The hit peptide list from the primary screen
(as identified in screen in NHPs and mice).

| | Peptide Amino | |
|---|---|---|
| Abbreviation | Acid Sequence | SEQ ID NO |
| EFS | EFSSNTVKLTS | 38 |
| SSN-L | GGSSNTVKLTSGHGG | 39 |
| SSN | SSNTVKLTSGH | 40 |
| SAN | SANFIKPTSY | 41 |
| VLT-L | GGVLTNIARGEYMRGG | 46 |
| IEI | IEINATRAGTNL | 42 |
| IEI-L | GGIEINATRAGTNLGG | 43 |

### Example 2. Secondary Validation

We followed up the primary screen in mice by generating GFP reporter vectors for several of the hit capsids. The vectors were injected at high dose IV to C57BL/6J mice. 2 weeks later, we necropsied the mice and collected GFP images of brain sections (data not shown). All of the hit vectors tested in the GFP study were de-localized from the liver, as evident from liver GFP staining (data not shown).

Higher magnification imaging revealed that the capsids SSN and SAN are significantly brain localized, but restricted to the endothelium. RCA-lectin is a co-stain marker of brain endothelial cells in these sections (data not shown). These results indicate that the identified AAV capsids in the screen are BBB-receptor binding, but do not cross the BBB. The mutant series showed a range of affinities for Ly6a receptor. Brain transduction levels decreased as correlated with observed tighter affinity binding of peptide to Ly6a receptor. The observed tight binding reduced transduction due to genomes likely stuck in the endothelium.

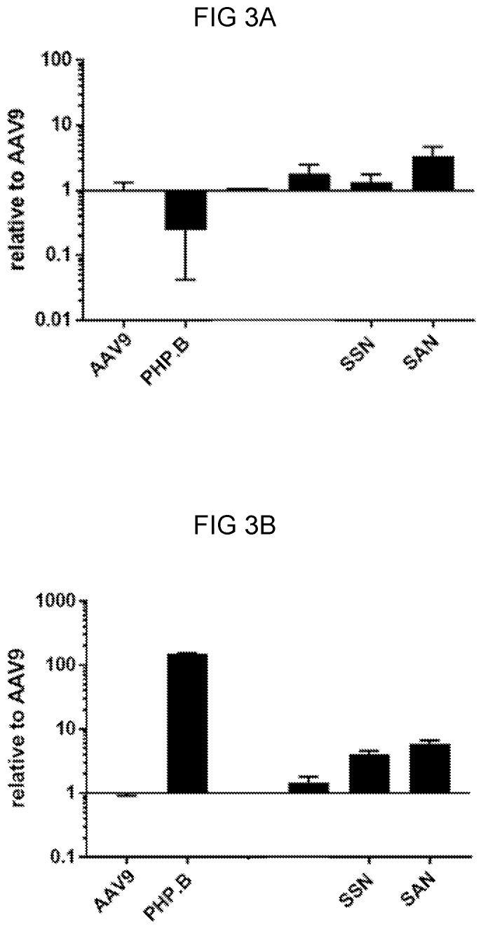

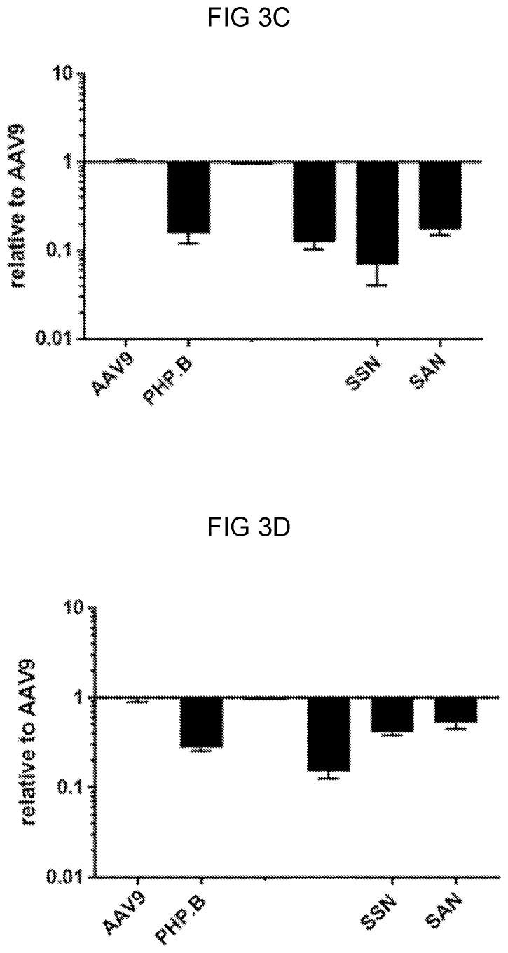

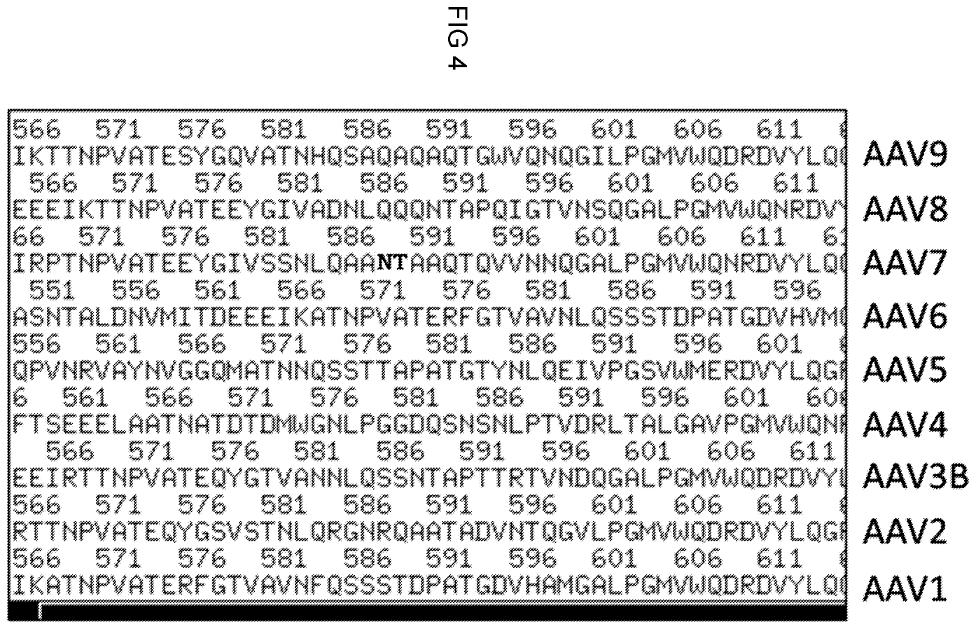

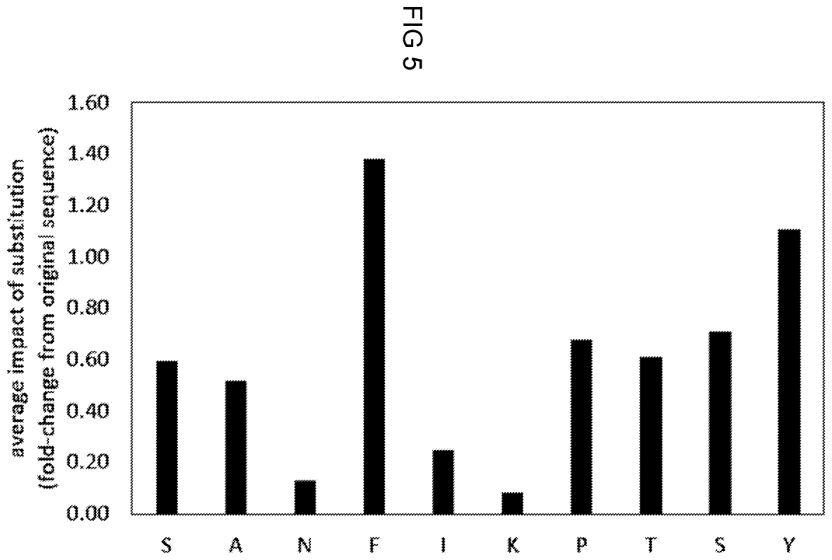

This endothelial localization may be useful for certain diseases. Also, this activity may be optimized to convert these from brain localizing to BBB-crossing vectors. We confirmed these BBB-crossing and brain-localizing activities in a barcoded vector study. Briefly, each capsid was used to individually produce vector containing a GFP reporter gene with a unique DNA barcode included. The barcoded capsid preps were mixed in equal proportions, and injected into C57BL/6J or Balb/c mice (FIG. 3A-3D). At the conclusion of in-life, the mouse tissues were subjected to NGS sequencing to count the abundance of each barcode among the vector genomes extracted from the tissue. The results confirm brain localization of vector genomes for all the hit capsids identified in the primary screen. In Balb/c mice, the secondary validation screen showed brain targeting for all hit sequences discovered in primary screen (FIG. 3A). In C576BL/6 mice the secondary validation screen showed brain targeting for all hit sequences discovered in primary screen (FIG. 3B). In both, Balb/c and C57BL/6 mice, liver de-targeting for all hit sequences, relative to AVA9 was consistent with affinity for brain vasculature (FIGS. 3C and 3D).

### Example 3. Endothelial Targeting Sequences

For NHP secondary validation, a barcode study was performed. The study was performed in two NHPs with injected mixture of 27 barcoded vectors, including AAV9 at 4.5×10¹³ GC/kg following 21-day in life. Analysis of a whole-brain tissue homogenate was performed. While some vectors showed a modestly improved vector biodistribution, no vectors showed an improved whole-brain transduction. The accumulation of vector genomes in the brain (DNA) and the expression of the vector-derived transcripts (mRNA) were poorly correlated (Table 2). We observed poor mRNA versus DNA correlation for endothelial-targeting vectors.

TABLE 2

| | |
|---|---|
| DNA | mRNA |

| | NHP1 | NHP2 | NHP1 | NHP2 |
|---|---|---|---|---|
| SSN | 7.2 | 4.2 | 0.1 | 0.1 |
| EFS | 7.0 | 3.8 | 0.1 | 0.1 |
| VLT-L | 4.4 | 1.6 | 0.1 | 0 |
| IEI-L | 1.7 | 4.2 | 0.1 | 0.1 |
| SSN-L | 2.9 | 2.3 | 0.1 | 0.1 |
| IEI | 2.8 | 1.9 | 0.1 | 0.1 |
| SAN | 2.8 | 1.9 | 0.1 | 0.1 |
| AAV9 | 1.0 | 1.0 | 1.0 | 1.0 |

Table 3, below, shows the average relative localization score for both of the NHPs in the barcode study, while normalized to AAV9 (equal to 1). In line with the brain-focused library design, most vectors have unremarkable localization in non-brain tissues. An exception to this is the vector PMK, which significantly re-localized to the spleen of both NHPs relative to AAV9.

TABLE 3

| AAV | Eye | Kidney | Liver | Lungs | Pancreas | Spleen | Testicle | Diaphr. | Quadrop | Heart |
|---|---|---|---|---|---|---|---|---|---|---|
| AAV9 | 1.0 | 1.0 | 1.0 | 1.0 | 1.0 | 1.0 | 1.0 | 1.0 | 1.0 | 1.0 |
| SSN | 1.5 | 0.3 | 0.3 | 0.3 | 0.2 | 0.0 | 0.4 | 1.1 | 1.0 | 0.6 |
| SSN-L | 1.3 | 0.2 | 0.5 | 0.2 | 0.2 | 0.1 | 0.6 | 0.9 | 0.9 | 0.6 |
| EFS | 2.6 | 0.4 | 0.4 | 0.7 | 0.2 | 0.1 | 0.9 | 1.6 | 1.4 | 1.0 |
| VLT-L | 0.9 | 0.2 | 0.5 | 0.3 | 0.2 | 0.1 | 0.4 | 0.6 | 0.6 | 0.4 |
| IEI | 0.6 | 0.2 | 0.1 | 0.5 | 0.1 | 0.0 | 0.2 | 0.3 | 0.5 | 0.2 |
| IEI-L | 1.0 | 0.2 | 0.1 | 0.4 | 0.1 | 0.6 | 0.8 | 0.5 | 0.7 | 0.5 |
| SAN | 1.0 | 0.3 | 0.3 | 0.5 | 0.1 | 0.1 | 0.3 | 0.7 | 0.8 | 0.4 |

Table 4, below, shows that brain endothelial hits have common in vitro transduction profiles (as measured in 293 transduction) and common production profiles. Importantly, vectors with the endothelial targeting activity all show dramatically increased relative abundance in the AAV vs Plasmid libraries as measured by NGS.

TABLE 4

| | | | | | Plasmid to | |
|---|---|---|---|---|---|---|
| | | | | | AAV | |
| Vector | Barcode | Barcide | | 293 | conversion | Vector |
| Name | DNA | mRNA | Insert Sequence | transd | in library | Yield |
| SSN | 5.7 | 0.1 | SSNTVKLTSGH | 20 | 54 | 0.9 |
| EFS | 5.4 | 0.1 | EFSSNTVKLTS | 17 | 35 | 0.8 |
| VLT-L | 3.0 | 0.0 | GGVLTNIARGEYMRGG | 21 | 16 | 0.6 |
| IEI-L | 3.0 | 0.1 | GGIEINATRAGTNLGG | 10 | 20 | 0.9 |
| SSN-L | 2.6 | 0.1 | GGSSNTVKLTSGHGG | 24 | 69 | 0.8 |
| IEI | 2.3 | 0.1 | IEINATRAGTNL | 10 | 14 | 0.9 |
| SAN | 2.3 | 0.1 | SANFIKPTSY | 43 | 26 | 0.7 |
| AAV9 | 1.0 | 1.0 | | 1 | | 1.0 |

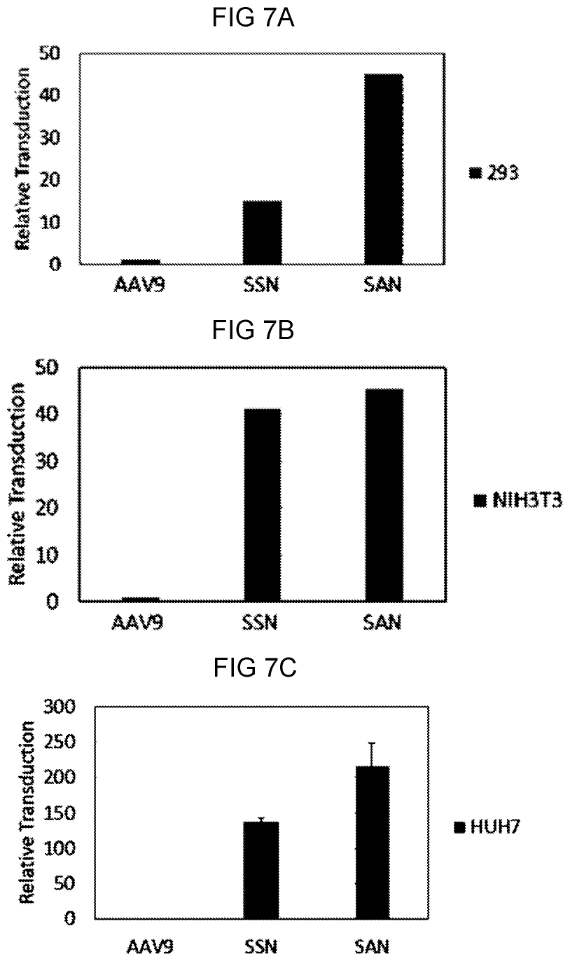

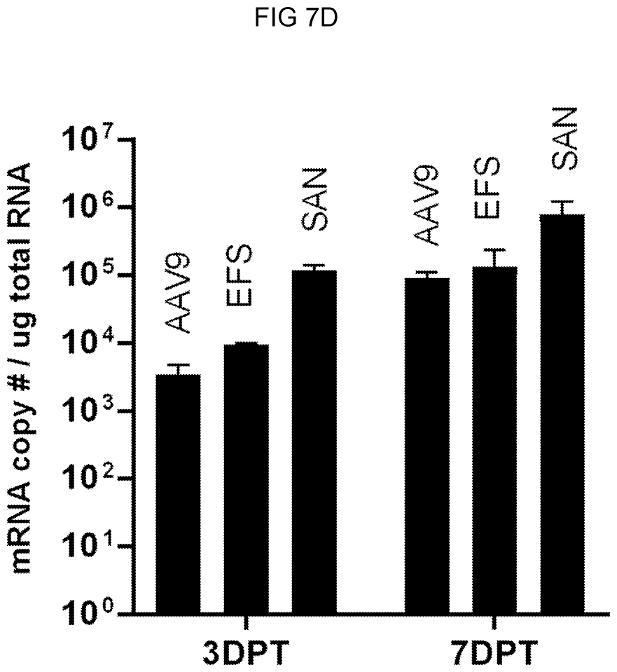

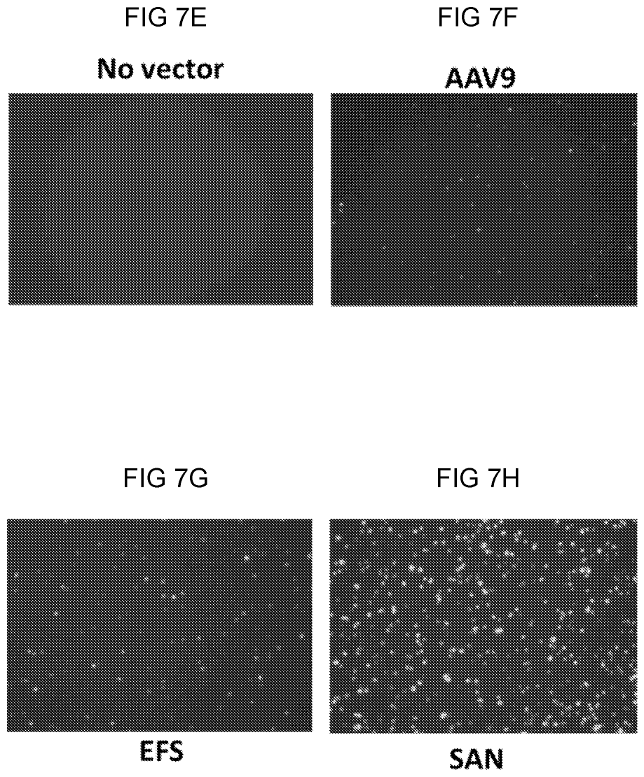

The mutation library of SAN peptide confirms the role of “NxTK” motif in brain targeting. In this study, every possible single amino acid change to SAN insert was made, the optimized library variants were injected in mice, and scores for biodistribution and yield for each variant was measured. “NxTK” motif is the critical motif for brain biodistribution in the SAN insert (Table 5 and FIG. 5). The “NxTK” motif controls plasmid-to-AAV conversion in the SAN peptide insert (Table 6 and FIG. 6). Furthermore, the “NxTK” motif linked three properties of these endothelial vectors in the “NxTK” class: the endothelial cell transduction, improved 293 cell transduction and ability to propagate during library production. FIGS. 7A to 7D show that “NxTK” motif confers broad transduction advantage across cell lines. The relative transduction levels were improved when compared to AAV9 capsid in 293 cells (FIG. 7A), NIH3T3 cells (FIG. 7B), and HUH7 cells (FIG. 7C). FIG. 7D shows a significant early improvement of transduction at day 3 post-transduction (3DPT) and approximately a 10-times improvement by day 7 post transduction (7DPT). AAV-GFP vectors with EFS and SAN peptide inserts showed an improved transduction when transduced the primary macaque airway epithelial cells (FIG. 7E-7H).

TABLE 5
Brain biodistribution (average from top to bottom).

| | S | A | N | F | I | K | P | T | S | Y |
|---|---|---|---|---|---|---|---|---|---|---|
| A | 0.4 | −0.4 | −4.8 | 1.0 | 0.3 | −3.8 | −0.3 | −0.5 | −0.2 | 0.1 |
| C | −6.0 | 0.0 | −4.0 | −1.5 | −4.6 | −3.1 | −4.0 | −4.0 | −3.1 | −1.1 |
| D | 0.7 | −1.0 | −3.7 | 0.5 | −2.6 | −8.3 | −1.2 | −0.8 | −0.3 | 0.7 |
| E | 0.0 | −0.8 | −2.1 | −0.5 | −4.2 | −5.3 | −1.8 | −0.4 | 0.2 | 0.9 |
| F | −1.3 | −2.4 | −1.6 | −0.4 | −3.9 | −1.9 | −1.4 | −0.9 | −2.2 | −0.2 |
| G | 0.1 | −0.9 | −2.7 | 1.3 | −3.1 | −4.6 | 0.5 | 0.4 | −0.2 | −0.7 |
| H | −1.8 | −1.0 | −4.3 | 0.2 | −2.4 | −3.0 | −1.1 | −1.7 | −1.9 | −1.3 |
| I | −1.0 | −0.9 | −5.2 | 0.8 | −0.4 | −3.5 | −0.9 | −1.2 | −0.8 | 0.2 |
| K | −4.3 | −2.8 | −4.9 | −0.4 | −3.5 | −4.0 | −1.2 | −2.1 | −3.2 | −2.7 |
| L | −0.6 | −0.8 | −3.8 | 0.2 | −3.7 | −3.3 | −1.1 | −1.1 | −0.5 | 0.4 |
| M | −0.7 | 0.2 | −4.2 | 0.6 | −3.0 | −4.1 | −0.8 | −0.8 | −0.5 | 0.4 |
| N | −0.3 | −0.7 | −0.4 | 1.0 | −1.1 | −4.4 | −0.2 | −0.5 | −0.7 | 0.5 |
| P | −1.1 | −0.1 | −4.5 | −2.5 | −2.3 | −3.6 | −0.4 | −0.8 | −0.2 | −0.2 |
| Q | 0.0 | −0.6 | −3.4 | 1.2 | −4.6 | −3.6 | −0.1 | −0.3 | −0.1 | 0.8 |
| R | −2.3 | −3.6 | −3.9 | −0.3 | −1.1 | −1.1 | −2.3 | −2.5 | −3.1 | −2.6 |
| S | −0.4 | −1.2 | −3.6 | 0.0 | −1.0 | −4.6 | −0.2 | −0.5 | −0.4 | 0.3 |
| T | 0.2 | −0.9 | −3.4 | 1.2 | 0.2 | −4.2 | −0.3 | −0.4 | −0.3 | 0.6 |
| V | −1.0 | −1.1 | −3.9 | 1.0 | −0.1 | −4.7 | −1.1 | −0.6 | −0.5 | 0.6 |
| W | −2.0 | −3.0 | 0.0 | −2.0 | −2.9 | −4.8 | 0.0 | −2.1 | 0.9 | −1.3 |
| Y | −1.6 | −4.8 | −1.9 | 0.0 | −4.4 | −4.1 | −1.1 | −1.4 | −0.8 | −0.4 |

TABLE 6
Plasmid to AAV yield conversion

| | S | A | N | F | I | K | P | T | S | Y |
|---|---|---|---|---|---|---|---|---|---|---|
| A | 0.6 | 0.4 | −1.1 | 1.1 | 1.3 | −1.4 | 1.3 | −0.1 | 0.4 | 1.2 |
| C | −3.1 | 11.0 | −6.2 | −2.6 | −6.0 | −6.8 | −4.3 | −5.4 | −4.4 | −1.7 |
| D | −0.1 | 0.6 | −0.7 | 0.3 | −0.7 | −1.1 | 1.0 | 0.2 | 0.8 | 0.9 |
| E | 0.3 | −1.2 | −1.0 | 0.7 | −0.6 | −0.6 | 0.9 | 0.1 | 0.8 | 0.8 |
| F | −2.8 | −5.5 | −5.1 | 0.4 | −3.7 | −7.2 | −5.5 | −3.7 | −3.2 | 0.2 |
| G | 0.4 | 0.7 | −0.9 | 1.2 | −0.4 | −0.5 | 1.2 | 0.9 | 0.7 | 1.1 |
| H | 0.2 | −0.9 | −1.6 | 1.3 | −0.6 | −1.7 | 1.0 | 0.0 | 0.1 | 0.9 |
| I | −0.9 | −1.4 | −2.9 | 1.1 | 0.4 | −3.3 | −1.9 | −0.7 | −0.9 | 1.0 |
| K | −0.5 | −0.5 | −1.3 | 0.3 | −1.6 | −3 | −1.2 | −2.5 | −1.8 | −1.1 |
| L | −0.5 | −1.1 | −2.6 | 1.1 | −1.1 | −4.5 | −1.7 | −1.2 | −1.2 | 0.6 |
| M | −0.6 | −1.5 | −2.4 | 1.3 | −0.6 | −3.0 | −0.4 | −0.9 | −0.8 | 1.0 |
| N | 1.0 | 0.5 | 0.4 | 1.5 | −0.1 | −1.0 | 1.5 | 0.6 | 0.7 | 1.2 |
| P | 0.7 | −0.2 | −1.1 | −0.4 | −0.4 | −0.9 | 0.4 | −0.4 | 0.4 | 0.7 |
| Q | 0.8 | 1.2 | −0.6 | 1.6 | −0.1 | −1.2 | 1.5 | 0.0 | 0.4 | 1.0 |
| R | −2.1 | −2.9 | −4.6 | −1.0 | −5.2 | −1.3 | −3.2 | −4.2 | −3.9 | −1.3 |
| S | 0.4 | 0.5 | −1.5 | 1.0 | 1.3 | −0.9 | 1.3 | 0.3 | 0.4 | 1.2 |
| T | 0.5 | 0.4 | −1.0 | 1.3 | 1.1 | −1.0 | 1.3 | 0.4 | 0.5 | 1.1 |
| V | 0.3 | −0.3 | −2.3 | 1.1 | 0.8 | −3.7 | −0.1 | −0.2 | 0.2 | 1.1 |
| W | −4.4 | −5.6 | −11.0 | −2.0 | −5.5 | −5.8 | −11.6 | −4.9 | −7.3 | −1.1 |
| Y | −1.8 | −2.9 | −6.3 | 0.2 | −3.8 | −6.8 | −4.3 | −2.9 | −3.1 | 0.4 |

In summary, the selected amino acid sequences, as listed in Table 7, all contain the functional motif N-x-(T/I/V/A)-(K/R) motif (SEQ ID NO: 47). Beyond the selected sequences shown in Table 7, others were identified during the screening. We have data that supports that many substitutions to these insert sequences also support or even improve the endothelial targeting activity. Additionally, we have discovered approximately thousands of sequences that fit this motif from large, random insert libraries—these sequences likely all share improved transduction properties.

TABLE 7
Selected Endothelial-Targeting Sequences

| Vector name | Insert sequence | SEQ ID NO |
|---|---|---|
| SSN | SSNTVKLTSGH | 40 |
| EFS | EFSSNTVKLTS | 38 |
| VLT-L | GGVLTNIARGEYMRGG | 46 |
| IEI-L | GGIEINATRAGTNLGG | 43 |
| SSN-L | GGSSNTVKLTSGHGG | 39 |
| IEI | IEINATRAGTNL | 42 |
| SAN | SANFIKPTSY | 41 |

We completed the barcode evaluation of primary screen hits in NHP. The brain localization is the most prominent feature of hits from this library, while there were no significant enhancements in targeting to peripheral tissues with possible exception of spleen targeting by AAV9-PMK. We defined a sequence motif common to all peptide inserts with brain-endothelial targeting activity. We confirmed the activity of this motif in brain-endothelial targeting as well as in conferring broad in vitro transduction advantage. A single sequence motif “NxTK” defines inserts from 4 unrelated sources that shares three properties:
(1) dramatically improved in vitro transduction on 293s and other cell lines;
(2) parasitic expansion during library production—“spreading” phenotype; and
(3) endothelial biodistribution in vivo in mice and NHP.

The “NxTK” motif was mapped in systemic mutational screen of SAN and EFS vector inserts. In systemic mutational screen, “NxTK” motif is shown to be critical for brain endothelial biodistribution as well as for abundance in library production. The plasmid-to-AAV conversion, meaning the capsid yield during library production, is controlled by 2 factors: presence of parasitic spreading motif (major factor) and intrinsic capsid yield (minor factor). One of the spreading motifs was identified to be “NxTK”, and was likely to interact with 293 cell-surface receptors and confer transduction advantage. The transduction advantage in 293 could lead to propagation of vector genome (Cap) to neighboring cells during production period since library production is done with limiting Cap, so most cells initially have everything except a Cap gene. The minor factor was only evident after digital filtering out of the vectors with parasitic spreading motif.

### Example 4. Engineering Strategy for AAV Capsid Development for Airway Delivery

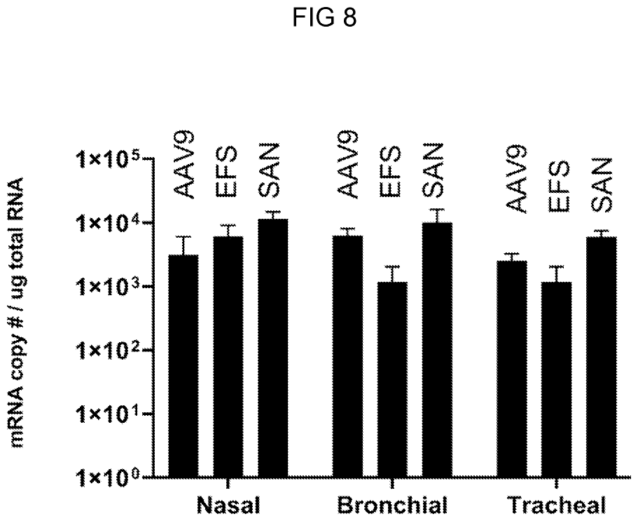

Current AAV vectors poorly transduce cells of the nasal passages and upper airway, limiting the efficacy of prophylactic AAV strategies to protect against upper airway infectious diseases like influenza or COVID-19. This project aims to engineer AAV capsids for improved transduction of these tissues, specifically a directed evolution and receptor targeting strategies are pursued. An engineering strategy pursued for AAV airway-specific capsid selection comprises of steps including but not limited to generating a diverse library of constructs with inserts (10³ to 10⁹ initial diversity), screening and selection in primary primate or human airway cells, identifying the genomic identity (DNA or RNA) of selected constructs, performing additional screening to converge on hits, validate hit of improved capsids in NHP. The sources for capsid diversity for airway delivery comprise of pursuing two approaches: unbiased and biased approach. In an unbiased approach, random peptides are inserted into AAV capsid surface to generate large libraries of greater than 10⁷ variant diversity. In biased approach, peptides with known or suspected airway-cell binding activities inserted into AAV Capsid to generate small libraries with approximately 10³ variant diversity. The source for such known or suspected airway-cell binding peptides: published phage display results, peptides from viral receptor binding domains, known ligands to airway receptors, and peptides generated in previous in vitro library screening. For screening of the generated library of capsid in vitro, an assay with primary airway cells in air-liquid interface (ALI) cultures is used. The cell used are of human and macaque origin, and comprise of nasal, trachea and bronchi cells origin. A preliminary transduction test with GFP vectors in macaque primary airway epithelial cell cultures showed a significant early improvement in transduction for EFS and SAN inserted peptides into AAV capsid (FIGS. 7D and 7E-7H). FIG. 7E shows microscopic analysis of the macaque primary airway epithelial cells in a control sample treated with carrier (i.e., no vector). FIG. 7F shows microscopic analysis of the macaque primary airway epithelial cells post-transduction with AAV9-GFP vector. FIG. 7G shows microscopic analysis of the macaque primary airway epithelial cells post-transduction with AAV9-GFP vector comprising EFS peptide insert. FIG. 7H shows microscopic analysis of the macaque primary airway epithelial cells post-transduction with AAV9-GFP comprising SAN peptide inserts. A preliminary transduction test with GFP vectors in cultured human cells showed overall lower transduction, wherein at day 7 the ration of mRNA copy number over micro-gram total mRNA was 1×10⁴ for cultured human cells (FIG. 8) and 1×10⁶ for cultured macaque primary airway epithelial cells (FIG. 7D). The SAN motif showed an advantage in transduction at Day 7 when in comparison to AAV9 transduction (FIG. 8). The EFS motif showed a poorer transduction in bronchial and tracheal cultured human cells (FIG. 8).

Furthermore, we have developed a number of insert sequences through in vitro selection schemes on other projects that confer dramatically improved cell binding and transduction activity. The generated AAV-insert vectors are tested in barcoded pools on ALI cultures. Results show that all in vitro selected AAV9-insert vectors were better than AAV9 vectors (results not shown). Specifically, AAV9-insert vectors of Spr3L (NxTK) were the best in comparison, the latter showing an approximately 50-fold better transduction than AAV9 capsid.

### Sequence Listing Free Text

The following information is provided for sequences containing free text under numeric identifier <223>.

| SEQ ID | |
|---|---|
| NO: | Free Text under <223> |
| 1 | <220> |
| | <223> AAV2/9 n.588.EFS nucleic acid sequence |
| | expression cassette |
| | <220> |
| | <221> misc_feature |
| | <222> (1) . . . (36) |
| | <223> truncated promoter |
| | <220> |
| | <221> promoter |
| | <222> (1) . . . (7) |
| | <223> p5 promoter |
| | <220> |
| | <221> CDS |
| | <222> (37) . . . (1899) |
| | <223> AAV2-Rep |
| | <220> |
| | <221> CDS |
| | <222> (1919) . . . (4162) |
| | <223> AAV9 Cap |
| | <220> |
| | <221> misc_feature |
| | <222> (3683) . . . (3715) |
| | <223> EFS |
| | <220> |
| | <221> misc_feature |
| | <222> (4253) . . . (4383) |
| | <223> p5 promoter |
| | <220> |
| | <221> misc_feature |
| | <222> (4511) . . . (4725) |
| | <223> LacZ promoter |
| 2 | <220> |
| | <223> Synthetic Construct |
| 3 | <220> |
| | <223> Synthetic Construct |
| 4 | <220> |
| | <223> AAV2/9 n.588.IEI nucleic acid sequence |
| | expression cassette |
| | <220> |
| | <221> misc_feature |
| | <222> (1) . . . (36) |
| | <223> truncated promoter |
| | <220> |
| | <221> promoter |
| | <222> (1) . . . (7) |
| | <223> p5 promoter |
| | <220> |
| | <221> CDS |
| | <222> (37) . . . (1899) |
| | <223> AAV2-Rep |
| | <220> |
| | <221> CDS |
| | <222> (1919) . . . (4165) |
| | <223> AAV9 Cap |
| | <220> |
| | <221> misc_feature |
| | <222> (3683) . . . (3718) |
| | <223> IEI |
| | <220> |
| | <221> misc_feature |
| | <222> (4256) . . . (4386) |
| | <223> p5 promoter |
| | <220> |
| | <221> misc_feature |
| | <222> (4514) . . . (4728) |
| | <223> LacZ promoter |
| 5 | <220> |
| | <223> Synthetic Construct |
| 6 | <220> |
| | <223> Synthetic Construct |
| 7 | <220> |
| | <223> AAV2/9 n.588.IEI-L nucleic acid sequence |
| | epxression cassette |
| | <220> |
| | <221> misc_feature |
| | <222> (1) . . . (36) |
| | <223> trunccated promoter |
| | <220> |
| | <221> promoter |
| | <222> (1) . . . (7) |
| | <223> p5 Promoter |
| | <220> |
| | <221> CDS |
| | <222> (37) . . . (1899) |
| | <223> AAV2 Rep |
| | <220> |
| | <221> CDS |
| | <222> (1919) . . . (4177) |
| | <223> AAV9 Cap |
| | <220> |
| | <221> misc_feature |
| | <222> (3683) . . . (3739) |
| | <223> IEI-L |
| | <220> |
| | <221> misc_feature |
| | <222> (4268) . . . (4398) |
| | <223> p5 promoter |
| | <220> |
| | <221> misc_feature |
| | <222> (4526) . . . (4740) |
| | <223> LacZ promoter |
| 8 | <220> |
| | <223> Synthetic Construct |
| 9 | <220> |
| | <223> Synthetic Construct |
| 10 | <210> 10 |
| | <211> 4722 |
| | <212> DNA |
| | <213> Artificial Sequence |
| | <220> |
| | <223> AAV2/9 n.588.SAN nucleic acid sequence |
| | expression cassette |
| | <220> |
| | <221> misc_feature |
| | <222> (1) . . . (36) |
| | <223> truncated promoter |
| | <220> |
| | <221> promoter |
| | <222> (1) . . . (7) |
| | <223> p5 promoter |
| | <220> |
| | <221> CDS |
| | <222> (37) . . . (1899) |
| | <223> AAV2 Rep |
| | <220> |
| | <221> CDS |
| | <222> (1919) . . . (4159) |
| | <223> AAV 9 Cap |
| | <220> |
| | <221> misc_feature |
| | <222> (3683) . . . (3712) |
| | <223> SAN |
| | <220> |
| | <221> misc_feature |
| | <222> (4250) . . . (4380) |
| | <223> p5 promoter |
| | <220> |
| | <221> misc_feature |
| | <222> (4508) . . . (4722) |
| | <223> LacZ promoter |
| 11 | <220> |
| | <223> Synthetic Construct |
| 12 | <220> |
| | <223> Synthetic Construct |
| 13 | <220> |
| | <223> AAV2/9 n.588.SSN nucleic acid sequence |
| | expression cassette |
| | <220> |
| | <221> misc_feature |
| | <222> (1) . . . (36) |
| | <223> trunctaed promoter |
| | <220> |
| | <221> promoter |
| | <222> (1) . . . (7) |
| | <223> p5 promoter |
| | <220> |
| | <221> CDS |
| | <222> (37) . . . (1899) |
| | <223> AAV2 Rep |
| | <220> |
| | <221> CDS |
| | <222> (1919) . . . (4162) |
| | <223> AAV9 Cap |
| | <220> |
| | <221> misc_feature |
| | <222> (3683) . . . (3715) |
| | <223> SSN |
| | <220> |
| | <221> misc_feature |
| | <222> (4253) . . . (4383) |
| | <223> p5 promoter |
| | <220> |
| | <221> misc_feature |
| | <222> (4511) . . . (4725) |
| | <223> LacZ promoter |
| 14 | <220> |
| | <223> Synthetic Construct |
| 15 | <220> |
| | <223> Synthetic Construct |
| 16 | <220> |
| | <223> AAV2/9 n.588.SSN-L nucleic acid sequence |
| | expression cassette |
| | <220> |
| | <221> misc_feature |
| | <222> (1) . . . (36) |
| | <223> truncated promoter |
| | <220> |
| | <221> promoter |
| | <222> (1) . . . (7) |
| | <223> p5 promoter |
| | <220> |
| | <221> CDS |
| | <222> (37) . . . (1899) |
| | <223> AAV2 Rep |
| | <220> |
| | <221> CDS |
| | <222> (1919) . . . (4174) |
| | <223> AAV9 Cap |
| | <220> |
| | <221> misc_feature |
| | <222> (3683) . . . (3727) |
| | <223> SSN-L |
| | <220> |
| | <221> misc_feature |
| | <222> (4253) . . . (4737) |
| | <223> LacZ promoter |
| | <220> |
| | <221> misc_feature |
| | <222> (4265) . . . (4395) |
| | <223> p5 promoter |
| 17 | <220> |
| | <223> Synthetic Construct |
| 18 | <220> |
| | <223> Synthetic Construct |
| 19 | <220> |
| | <223> AAV2/9 n.588.VLT-L nucleic acid sequence |
| | expression cassette |
| | <220> |
| | <221> misc_feature |
| | <222> (1) . . . (36) |
| | <223> truncated promoter |
| | <220> |
| | <221> promoter |
| | <222> (1) . . . (7) |
| | <223> p5 promoter |
| | <220> |
| | <221> CDS |
| | <222> (37) . . . (1899) |
| | <223> AAV2 Rep |
| | <220> |
| | <221> CDS |
| | <222> (1919) . . . (4177) |
| | <223> AAV9 Cap |
| | <220> |
| | <221> misc_feature |
| | <222> (3683) . . . (3730) |
| | <223> VLT-L |
| | <220> |
| | <221> misc_feature |
| | <222> (4268) . . . (4398) |
| | <223> p5 promoter |
| | <220> |
| | <221> misc_feature |
| | <222> (4526) . . . (4740) |
| | <223> LacZ promoter |
| 20 | <220> |
| | <223> Synthetic Construct |
| 21 | <220> |
| | <223> Synthetic Construct |
| 22 | <220> |
| | <223> AAV2 Rep nucleic acid sequence |
| 23 | <220> |
| | <223> AAV2 Rep amino acid sequence |
| 24 | <220> |
| | <223> AAV9 Cap n.588.EFS nucleic acid sequence |
| | <220> |
| | <221> misc_feature |
| | <222> (1765) . . . (1797) |
| | <223> EFS |
| 25 | <220> |
| | <223> AAV9 Cap n.588.EFS amino acid sequence |
| | <220> |
| | <221> MISC_FEATURE |
| | <222> (499) . . . (599) |
| | <223> EFS |
| 25 | <220> |
| | <223> AAV9 Cap n588.IEI nucleic acid sequence |
| | <220> |
| | <221> misc_feature |
| | <222> (1765) . . . (1800) |
| | <223> IEI |
| 27 | <220> |
| | <223> AAV9 Cap n588.IEI amino acid sequence |
| | <220> |
| | <221> MISC_FEATURE |
| | <222> (499) . . . (600) |
| | <223> IEI |
| 28 | <220> |
| | <223> AAV9 Cap n.588.IEI-L nucleic acid |
| | sequence |
| | <220> |
| | <221> misc_feature |
| | <222> (1765) . . . (1812) |
| | <223> IEI-L |
| 29 | <220> |
| | <223> AAV9 Cap n.588.IEI-L amino acid |
| | sequence |
| | <220> |
| | <221> MISC_FEATURE |
| | <222> (499) . . . (604) |
| | <223> IEI-L |
| 30 | <220> |
| | <223> AAV9 Cap n.588.SAN nucleic acid |
| | sequence |
| | <220> |
| | <221> misc_feature |
| | <222> (1765) . . . (1794) |
| | <223> SAN |
| 31 | <220> |
| | <223> AAV9 Cap n.588.SAN amino acid |
| | sequence |
| | <220> |
| | <221> MISC_FEATURE |
| | <222> (499) . . . (598) |
| | <223> SAN |
| 32 | <220> |
| | <223> AAV9 Cap n.588.SSN nucleic acid |
| | sequence |
| | <220> |
| | <221> misc_feature |
| | <222> (1765) . . . (1797) |
| | <223> SSN |
| 33 | <220> |
| | <223> AAV9 Cap n.588.SSN amino acid sequence |
| | <220> |
| | <221> MISC_FEATURE |
| | <222> (499) . . . (599) |
| | <223> SSN |
| 34 | <220> |
| | <223> AAV9 Cap n.588.SSN-L nucleic acid |
| | sequence |
| | <220> |
| | <221> misc_feature |
| | <222> (1765) . . . (1809) |
| | <223> SSN-L |
| 35 | <220> |
| | <223> AAV9 Cap n.588.SSN-L amino acid |
| | sequence |
| | <220> |
| | <221> MISC_FEATURE |
| | <222> (499) . . . (603) |
| | <223> SSN-L |
| 36 | <220> |
| | <223> AAV9 Cap n588.VLT-L nucleic acid |
| | sequence |
| | <220> |
| | <221> misc_feature |
| | <222> (1765) . . . (1812) |
| | <223> VLT-L |
| 37 | <220> |
| | <223> AAV9 Cap n588. VLT-L amino acid |
| | sequence |
| | <220> |
| | <221> MISC_FEATURE |
| | <222> (499) . . . (604) |
| | <223> VLT-L |
| 38 | <220> |
| | <223> EFS peptide sequence |
| 39 | <220> |
| | <223> SSN-L peptide sequence |
| 40 | <220> |
| | <223> SSN peptide sequence |
| 41 | <220> |
| | <223> SAN peptide sequence |
| 42 | <220> |
| | <223> IEI peptide sequence |
| 43 | <220> |
| | <223> IEI-L peptide sequence |
| 44 | <220> |
| | <223> AAV9 capsid |
| 45 | <220> |
| | <223> AAVhu68 capsid |
| 46 | <220> |
| | <223> VLT-L peptide sequence |
| 47 | <220> |
| | <223> N-X-(T/I/V/A)-(K/R) motif |
| | <220> |
| | <221> MISC_FEATURE |
| | <222> (2) . . . (2) |
| | <223> any amino acid |
| | <220> |
| | <221> MISC_FEATURE |
| | <222> (3) . . . (3) |
| | <223> Xaa is selected from Threonine (T), |
| | Isoleucine (I), Valine (V) or Alanine (A) |
| | <220> |
| | <221> MISC_FEATURE |
| | <222> (4) . . . (4) |
| | <223> Xaa is selected from Lysine (K) or Arginine (R) |
| 48 | <220> |
| | <223> AAV2 variant peptide NDVRAVS |
| 49 | <220> |
| | <223> PHP.B peptide insert |
| 50 | <220> |
| | <223> nucleic acid sequence EFS |
| 51 | <220> |
| | <223> nucleic acid sequence IEI |
| 52 | <220> |
| | <223> nucleic acid sequence IEI-L |
| 53 | <220> |
| | <223> nucleic acid sequence SAN |
| 54 | <220> |
| | <223> nucleic acid sequence SSN |
| 55 | <220> |
| | <223> nucleic acid sequence SSN-L |
| 56 | <220> |
| | <223> nucleic acid sequence VLT-L |

All documents cited in this specification are incorporated herein by reference. U.S. Provisional Application No. 63/119,863, filed Dec. 1, 2020 is incorporated herein by reference in its entirety. The sequence listing filed herewith named “20-9409PCT_ST25” and the sequences and text therein are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated virus (AAV) particle (rAAV) having an AAV capsid comprising an amino acid sequence that comprises a motif N-x-(T/I/V/A)-(K/R) (SEQ ID NO: 47), wherein the amino acid sequence is part of at least the AAV vp3 protein in the AAV capsid, and wherein the rAAV comprises a vector genome packaged in the AAV capsid, wherein the vector genome comprises a nucleic acid sequence encoding a gene product under control of sequences which direct expression thereof, provided that the AAV capsid is not a mutant AAV2 capsid comprising an NDVRAVS (SEQ ID NO: 48) sequence.

2. The rAAV of claim 1, wherein the amino acid sequence comprising—the N-x-(T/I/V/A)-(K/R) motif is optionally flanked at the amino terminus and/or the carboxy terminus of the motif by two amino acids to seven amino acids, and is inserted into the AAV capsid vp3 region.

3. The rAAV of claim 1, wherein the motif sequence inserted into the AAV capsid comprises:
(a)
(SEQ ID NO: 40)
SSNTVKLTSGH;
(b)
(SEQ ID NO: 38)
EFSSNTVKLTS;
(c)
(SEQ ID NO: 46)
GGVLTNIARGEYMRGG;
(d)
(SEQ ID NO: 43)
GGIEINATRAGTNLGG;
(e)
(SEQ ID NO: 39)
GGSSNTVKLTSGHGG;
(f)
(SEQ ID NO: 42)
IEINATRAGTNL;
or
(g)
(SEQ ID NO: 41)
SANFIKPTSY.

4. The rAAV of claim 1, wherein the amino acid sequence of the motif is NTVK.

5. The rAAV of claim 1, wherein the motif N-x-(T/I/V/A)-(K/R) (SEQ ID NO: 47) is optionally flanked by two to seven amino acids at its carboxy- and/or amino terminus and inserted between amino acids 588 and 589 of an AAV9 capsid protein, based on the numbering of amino acid sequence: SEQ ID NO: 44.

6. A composition comprising a stock of the rAAV of claim 1 and one or more of a physiologically compatible carrier, excipient, and/or aqueous suspension base.

7. An endothelial cell targeting peptide, wherein the endothelial cell targeting peptide comprises a motif comprising an amino acid sequence of N-x-(T/I/V/A)-(K/R) (SEQ ID NO: 47) optionally flanked at the amino terminus and/or the carboxy terminus of the motif by two amino acids to seven amino acids, and optionally further conjugated to a nanoparticle, a second molecule, or a viral capsid protein.

8. The endothelial cell targeting peptide of claim 7, wherein the endothelial cell targeting peptide comprises:
(a)
(SEQ ID NO: 40)
SSNTVKLTSGH;
(b)
(SEQ ID NO: 38)
EFSSNTVKLTS;
(c)
(SEQ ID NO: 46)
GGVLTNIARGEYMRGG;
(d)
(SEQ ID NO: 43)
GGIEINATRAGTNLGG;
(e)
(SEQ ID NO: 39)
GGSSNTVKLTSGHGG;
(f)
(SEQ ID NO: 42)
IEINATRAGTNL;
or
(g)
(SEQ ID NO: 41)
SANFIKPTSY.

9. The endothelial cell targeting peptide of claim 7, wherein the amino acid sequence of the motif is NTVK.

10. A composition comprising the endothelial cell targeting peptide of claim 7 and one or more of a physiologically compatible carrier, excipient, and/or aqueous suspension base.

11. A fusion polypeptide or protein comprising a brain endothelial cell targeting peptide of claim 7 and a fusion partner which comprises at least one polypeptide or protein.

12. A composition comprising a fusion polypeptide or protein of claim 11 and one or more of a physiologically compatible carrier, excipient, and/or aqueous suspension base.

13. (canceled)

14. A method for targeting therapy to the endothelial cells in brain, said method comprising administering to a patient in need thereof a stock of rAAV of claim 1.

15. A method for treating Allan-Herndon-Dudley disease by delivering to a subject in need thereof a stock of rAAV of claim 1, wherein the encoded gene product is an MCT8 protein.

16. A method for targeting therapy to the lung comprising administering to a patient in need thereof a stock of rAAV of claim 1.

17. A method for treating a disease of the lung by delivering to a subject in need thereof a stock of rAAV of claim 1, wherein the encoded gene product is a soluble Ace2 protein, an anti-SARS antibody, an anti-SARS-CoV2 antibody, an anti-influenza antibody, or a cystic fibrosis transmembrane protein.

18. A method for increasing transduction of AAV production cells in vitro comprising inserting an N-x-(T/I/V/A)-(K/R) (SEQ ID NO: 47) motif into an AAV capsid.

19. The method of claim 16, wherein the production cells are 293 cells.


SEQUENCE LISTING (56 sequences)

SEQ ID NO: 1 - DNA, 4725 bp, Artificial Sequence
  AAV2/9 n.588.EFS nucleic acid sequence expression cassette
  misc_feature (1)..(36): truncated promoter
  promoter (1)..(7): p5 promoter
  CDS (37)..(1899): AAV2-Rep
  CDS (1919)..(4162): AAV9 Cap
  misc_feature (3683)..(3715): EFS
  misc_feature (4253)..(4383): p5 promoter
  misc_feature (4511)..(4725): LacZ promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagagttcagcagcaacaccgtgaagctgaccagcgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 2 - protein, 621 aa, Artificial Sequence
  Synthetic Construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 3 - protein, 747 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQEFSSNTVKLTSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - DNA, 4728 bp, Artificial Sequence
  AAV2/9 n.588.IEI nucleic acid sequence expression cassette
  misc_feature (1)..(36): truncated promoter
  promoter (1)..(7): p5 promoter
  CDS (37)..(1899): AAV2-Rep
  CDS (1919)..(4165): AAV9 Cap
  misc_feature (3683)..(3718): IEI
  misc_feature (4256)..(4386): p5 promoter
  misc_feature (4514)..(4728): LacZ promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaatcgagatcaacgctaccagagctggaaccaacctggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 5 - protein, 621 aa, Artificial Sequence; identical to SEQ ID NO: 2
  Synthetic Construct

SEQ ID NO: 6 - protein, 748 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQIEINATRAGTNLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - DNA, 4740 bp, Artificial Sequence
  AAV2/9 n.588.IEI-L nucleic acid sequence epxression cassette
  misc_feature (1)..(36): trunccated promoter
  promoter (1)..(7): p5 Promoter
  CDS (37)..(1899): AAV2 Rep
  CDS (1919)..(4177): AAV9 Cap
  misc_feature (3683)..(3739): IEI-L
  misc_feature (4268)..(4398): p5 promoter
  misc_feature (4526)..(4740): LacZ promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaggaggaatcgagatcaacgctaccagagctggaaccaacctgggaggagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 8 - protein, 621 aa, Artificial Sequence; identical to SEQ ID NO: 2
  Synthetic Construct

SEQ ID NO: 9 - protein, 752 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGGIEINATRAGTNLGGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - DNA, 4722 bp, Artificial Sequence
  AAV2/9 n.588.SAN nucleic acid sequence expression cassette
  misc_feature (1)..(36): truncated promoter
  promoter (1)..(7): p5 promoter
  CDS (37)..(1899): AAV2 Rep
  CDS (1919)..(4159): AAV 9 Cap
  misc_feature (3683)..(3712): SAN
  misc_feature (4250)..(4380): p5 promoter
  misc_feature (4508)..(4722): LacZ promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagcgctaacttcatcaagcctaccagctacgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 11 - protein, 621 aa, Artificial Sequence; identical to SEQ ID NO: 2
  Synthetic Construct

SEQ ID NO: 12 - protein, 746 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSANFIKPTSYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - DNA, 4725 bp, Artificial Sequence
  AAV2/9 n.588.SSN nucleic acid sequence expression cassette
  misc_feature (1)..(36): trunctaed promoter
  promoter (1)..(7): p5 promoter
  CDS (37)..(1899): AAV2  Rep
  CDS (1919)..(4162): AAV9 Cap
  misc_feature (3683)..(3715): SSN
  misc_feature (4253)..(4383): p5 promoter
  misc_feature (4511)..(4725): LacZ promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagcagcaacaccgtgaagctgaccagcggacacgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 14 - protein, 621 aa, Artificial Sequence; identical to SEQ ID NO: 2
  Synthetic Construct

SEQ ID NO: 15 - protein, 747 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSSNTVKLTSGHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - DNA, 4737 bp, Artificial Sequence
  AAV2/9 n.588.SSN-L nucleic acid sequence expression cassette
  misc_feature (1)..(36): truncated promoter
  promoter (1)..(7): p5 promoter
  CDS (37)..(1899): AAV2 Rep
  CDS (1919)..(4174): AAV9 Cap
  misc_feature (3683)..(3727): SSN-L
  misc_feature (4253)..(4737): LacZ promoter
  misc_feature (4265)..(4395): p5 promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaggaggaagcagcaacaccgtgaagctgaccagcggacacggaggagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 17 - protein, 621 aa, Artificial Sequence; identical to SEQ ID NO: 2
  Synthetic Construct

SEQ ID NO: 18 - protein, 751 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGGSSNTVKLTSGHGGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - DNA, 4740 bp, Artificial Sequence
  AAV2/9 n.588.VLT-L nucleic acid sequence expression cassette
  misc_feature (1)..(36): truncated promoter
  promoter (1)..(7): p5 promoter
  CDS (37)..(1899): AAV2 Rep
  CDS (1919)..(4177): AAV9 Cap
  misc_feature (3683)..(3730): VLT-L
  misc_feature (4268)..(4398): p5 promoter
  misc_feature (4526)..(4740): LacZ promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaggaggagtgctgaccaacatcgctagaggagagtacatgagaggaggagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 20 - protein, 621 aa, Artificial Sequence; identical to SEQ ID NO: 2
  Synthetic Construct

SEQ ID NO: 21 - protein, 752 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGGVLTNIARGEYMRGGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - DNA, 1863 bp, Artificial Sequence
  AAV2 Rep nucleic acid sequence
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 23 - protein, 621 aa, Artificial Sequence; identical to SEQ ID NO: 2
  AAV2 Rep amino acid sequence

SEQ ID NO: 24 - DNA, 2244 bp, Artificial Sequence
  AAV9 Cap n.588.EFS nucleic acid sequence
  misc_feature (1765)..(1797): EFS
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagagttcagcagcaacaccgtgaagctgaccagcgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 25 - protein, 747 aa, Artificial Sequence; identical to SEQ ID NO: 3
  AAV9 Cap n.588.EFS amino acid sequence
  MISC_FEATURE (499)..(599): EFS

SEQ ID NO: 26 - DNA, 2247 bp, Artificial Sequence
  AAV9 Cap n588.IEI nucleic acid sequence
  misc_feature (1765)..(1800): IEI
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaatcgagatcaacgctaccagagctggaaccaacctggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 27 - protein, 748 aa, Artificial Sequence; identical to SEQ ID NO: 6
  AAV9 Cap n588.IEI amino acid sequence
  MISC_FEATURE (499)..(600): IEI

SEQ ID NO: 28 - DNA, 2259 bp, Artificial Sequence
  AAV9 Cap n.588.IEI-L nucleic acid sequence
  misc_feature (1765)..(1812): IEI-L
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaggaggaatcgagatcaacgctaccagagctggaaccaacctgggaggagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 29 - protein, 752 aa, Artificial Sequence; identical to SEQ ID NO: 9
  AAV9 Cap n.588.IEI-L amino acid sequence
  MISC_FEATURE (499)..(604): IEI-L

SEQ ID NO: 30 - DNA, 2241 bp, Artificial Sequence
  AAV9 Cap n.588.SAN nucleic acid sequence
  misc_feature (1765)..(1794): SAN
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagcgctaacttcatcaagcctaccagctacgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 31 - protein, 746 aa, Artificial Sequence; identical to SEQ ID NO: 12
  AAV9 Cap n.588.SAN amino acid sequence
  MISC_FEATURE (499)..(598): SAN

SEQ ID NO: 32 - DNA, 2244 bp, Artificial Sequence
  AAV9 Cap n.588.SSN nucleic acid sequence
  misc_feature (1765)..(1797): SSN
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagcagcaacaccgtgaagctgaccagcggacacgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 33 - protein, 747 aa, Artificial Sequence; identical to SEQ ID NO: 15
  AAV9 Cap n.588.SSN amino acid sequence
  MISC_FEATURE (499)..(599): SSN

SEQ ID NO: 34 - DNA, 2256 bp, Artificial Sequence
  AAV9 Cap n.588.SSN-L nucleic acid sequence
  misc_feature (1765)..(1809): SSN-L
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaggaggaagcagcaacaccgtgaagctgaccagcggacacggaggagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 35 - protein, 751 aa, Artificial Sequence; identical to SEQ ID NO: 18
  AAV9 Cap n.588.SSN-L amino acid sequence
  MISC_FEATURE (499)..(603): SSN-L

SEQ ID NO: 36 - DNA, 2259 bp, Artificial Sequence
  AAV9 Cap n588.VLT-L nucleic acid sequence
  misc_feature (1765)..(1812): VLT-L
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaggaggagtgctgaccaacatcgctagaggagagtacatgagaggaggagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 37 - protein, 752 aa, Artificial Sequence; identical to SEQ ID NO: 21
  AAV9 Cap n588.VLT-L amino acid sequence
  MISC_FEATURE (499)..(604): VLT-L

SEQ ID NO: 38 - protein, 11 aa, Artificial Sequence
  EFS peptide sequence
EFSSNTVKLTS

SEQ ID NO: 39 - protein, 15 aa, Artificial Sequence
  SSN-L peptide sequence
GGSSNTVKLTSGHGG

SEQ ID NO: 40 - protein, 11 aa, Artificial Sequence
  SSN peptide sequence
SSNTVKLTSGH

SEQ ID NO: 41 - protein, 10 aa, Artificial Sequence
  SAN peptide sequence
SANFIKPTSY

SEQ ID NO: 42 - protein, 12 aa, Artificial Sequence
  IEI peptide sequence
IEINATRAGTNL

SEQ ID NO: 43 - protein, 16 aa, Artificial Sequence
  IEI-L peptide sequence
GGIEINATRAGTNLGG

SEQ ID NO: 44 - protein, 736 aa, Artificial Sequence
  AAV9 capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 736 aa, Artificial Sequence
  AAVhu68 capsid
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 16 aa, Artificial Sequence
  VLT-L peptide sequence
GGVLTNIARGEYMRGG

SEQ ID NO: 47 - protein, 4 aa, Artificial Sequence
  N-X-(T/I/V/A)-(K/R) motif
  MISC_FEATURE (2)..(2): any amino acid
  MISC_FEATURE (3)..(3): Xaa is selected from Threonine (T), Isoleucine (I), Valine (V) or Alanine (A)
  MISC_FEATURE (4)..(4): Xaa is selelcted from Lysine (K) or Arginine (R)
NXXX

SEQ ID NO: 48 - protein, 7 aa, Artificial Sequence
  AAV2 variant peptide NDVRAVS
NDVRAVS

SEQ ID NO: 49 - protein, 7 aa, Artificial Sequence
  PHP.B peptide insert
TLAVPFK

SEQ ID NO: 50 - DNA, 33 bp, Artificial Sequence
  nucleic acid sequence EFS
gagttcagcagcaacaccgtgaagctgaccagc

SEQ ID NO: 51 - DNA, 36 bp, Artificial Sequence
  nucleic acid sequence IEI
atcgagatcaacgctaccagagctggaaccaacctg

SEQ ID NO: 52 - DNA, 48 bp, Artificial Sequence
  nucleic acid sequence IEI-L
ggaggaatcgagatcaacgctaccagagctggaaccaacctgggagga

SEQ ID NO: 53 - DNA, 30 bp, Artificial Sequence
  nucleic acid sequence SAN
agcgctaacttcatcaagcctaccagctac

SEQ ID NO: 54 - DNA, 33 bp, Artificial Sequence
  nucleic acid sequence SSN
agcagcaacaccgtgaagctgaccagcggacac

SEQ ID NO: 55 - DNA, 45 bp, Artificial Sequence
  nucleic acid sequence SSN-L
ggaggaagcagcaacaccgtgaagctgaccagcggacacggagga

SEQ ID NO: 56 - DNA, 48 bp, Artificial Sequence
  nucleic acid sequence VLT-L
ggaggagtgctgaccaacatcgctagaggagagtacatgagaggagga